(19) United States
(12) Patent Application Publication
(10) Pub. No.: US 2020/0300842 A1
(43) Pub. Date: Sep. 24, 2020

(54) ELECTRONIC SORBENT ASSAY

(71) Applicant: EPITRONIC HOLDINGS PTE. LTD., Singapore (SG)

(72) Inventors: Ayal RAM, Singapore (SG); Walid-Madhat MUNIEF, Blieskastel (DE)

(21) Appl. No.: 16/818,039

(22) Filed: Mar. 13, 2020

Related U.S. Application Data
(60) Provisional application No. 62/821,882, filed on Mar. 21, 2019.

(51) Int. Cl. G01N 33/538; G01N 33/49; G01N 15/14; H01L 29/778; H01L 29/20

(52) U.S. Cl. CPC G01N 33/538; G01N 33/4915; H01L 29/2003; H01L 29/778; G01N 15/1404

(57) ABSTRACT

The present invention provides a microelectronic device for sorbent, immunosorbent or cell sorbent assay, or flow cytometry, and for measuring biological cell dynamics. The device is based on an open-gate pseudo-conductive high-electron mobility transistor, which is based on a multilayer hetero-junction structure being made of III-V single- or polycrystalline semi-conductor materials and deposited on a substrate layer or placed on a free-standing membrane. Said structure comprising at least one buffer layer and at least one barrier layer, said layers being stacked alternately, wherein the thickness of a top (barrier or buffer) layer in an open gate area of said transistor is 5-9 nanometre (nm), which corresponds to the pseudo-conducting current range between normally-on and normally-off operation mode of the transistor, and the surface of said top layer has a roughness of about 0.2 nm or less. In addition, the present invention provides methods for sorbent, immunosorbent or cell sorbent assay and for measuring biological cell dynamics using said device.

### CROSS REFERENCE TO RELATED PARAGRAPHS

This application claims priority to U.S. 62/821,882 entitled ELECTRONIC SORBENT ASSAY filed on Mar. 21, 2019. The contents of the above applications are all incorporated by reference as if fully set forth herein in their entirety.

### TECHNICAL FIELD OF THE INVENTION

The present application relates to the field of microelectronic sensors based on open-gate pseudo-conductive high-electron-mobility transistors (PC-HEMT) and their use in sorbent, immunosorbent and sorbent cell assays and for measuring biological cell dynamics.

### BACKGROUND OF THE INVENTION

### High-Electron Mobility Transistors

The polarization doped high-electron-mobility transistor (HEMT) is a field effect transistor (FET) in which two layers of different bandgap and polarisation field are grown upon each other forming a hetero-junction structure. As a consequence of the discontinuity in the polarisation field, surface charges are created at the interface between the layers of the hetero junction structure. If the induced surface charge is positive, electrons will tend to compensate the induced charge resulting in the formation of the channel. Since in the HEMT, the channel electrons are confined in a quantum well in an infinitely narrow spatial region at the interface between the layers, these electrons are referred to as a two-dimensional electron gas (2DEG). This special confinement of the channel electrons in the quantum well actually grants them two-dimensional features, which strongly enhance their mobility surpassing the bulk mobility of the material in which the electrons are flowing.

The HEMTs based on the layers of III-V semiconductor materials, such as gallium nitride (GaN) and aluminium gallium nitride (AlGaN), have recently been developed with a view to high-voltage and high-power switching applications. The high voltages and high switching speeds allow smaller, more efficient devices, such as home appliances, communications and automobiles to be manufactured. To control the density of electrons in the 2DEG channel and to switch the HEMT on and off, the voltage at the gate of the transistor should be regulated.

FIGS. 1a-1c schematically shows the quantum well at three different biasing conditions starting from the positive gate potential (V_{G}), much higher than the threshold voltage (V_{T}), and going down to the 0V gate potential and further to the negative values below the threshold voltage. The V_{T} is defined as a voltage required to populate electrons at the interface between the GaN and AlGaN layers, thereby creating conductivity of the 2DEG channel. Since the 2DEG channel electrons occupy energy levels below the Fermi level, the Fermi level in a quantum well is located above several energy levels when V_{G}>>V_{T} (FIG. 1a). This enables high population of the 2DEG channel electrons and hence, high conductivity. The HEMT is turned on in this case. However, when V_{G} decreases to 0V (FIG. 1b), the Fermi level also drops with respect to the quantum well. As a result, much fewer electron energy levels are populated and the amount of the 2DEG channel electrons significantly decreases. When V_{G}<<V_{T} (FIG. 1c), all electron energy levels are above the Fermi level, and there is no 2DEG electrons below the gate. This situation is called “channel depletion”, and the HEMT is turned off.

Many commercially available AlGaN/GaN-based HEMT structures have a negative V_{T}, resulting in a “normally-on” operation mode at 0V gate potential. They are called “depletion-mode transistors” and used in various power switching applications when the negative voltage must be applied on the gate in order to block the current. However, for safe operation at high voltage or high-power density, in order to reduce the circuit complexity and eliminate standby power consumption, HEMTs with “normally-off” characteristics are preferred.

Several techniques to manufacture the normally-off HEMTs have been reported. Burnham et al in “Gate-recessed normally-off GaN-on-Si HEMT using a new O₂-BCl₃ digital etching technique”, Phys. Status Solidi C, Vol. 7, 2010, No. 7-8, pp. 2010-2012, proposed normally-off structures of the recessed gate type. In this structure, the AlGaN barrier layer is etched and the gate is brought closer to the interface between the AlGaN barrier layer and the GaN buffer layer. As the gate approaches the interface between the layers, the V_{T} increases. The normally-off operation of the transistor is achieved once the depletion region reaches the interface and depletes the 2DEG channel at zero gate voltage. The major advantages of these HEMTs are lower power consumption, lower noise and simpler drive circuits. These HEMTs are currently used, for example, in microwave and millimetre wave communications, imaging and radars.

Chang et al in “Development of enhancement mode AlN/GaN high electron mobility transistors”, Appl. Phys. Lett., Vol. 94, 2009, No. 26, p. 263505, proposed using a very thin AlGaN barrier instead of etching the relatively thick barrier layer to approach the AlGaN/GaN interface. This structure also achieves the normally-off operation by approaching the transistor gate towards the AlGaN/GaN interface. Chen et al (2010) in “Self-aligned enhancement-mode AlGaN/GaN HEMTs using 25 keV fluorine ion implantation”, in Device Research Conference (DRC), 2010, pp. 137-138, proposed to use the fluorine-based plasma treatment method. Although many publications have adopted various methods to achieve normally-off devices with minimum impact on the drain current, they unfortunately sacrificed device turn-on performance.

### Sorbent Assays

Sorbent assays, including enzyme-linked immunosorbent assay (ELISA) and molecularly-imprinted sorbent assays, are plate-based assays designed for detecting and quantifying chemical and biochemical substances such as peptides, proteins, antibodies hormones and small organic molecules. In a traditional ELISA, an antigen is immobilised to a solid surface and then conjugated to an antibody that is linked to an enzyme. There are three types of ELISA, where each type of the assay can be used qualitatively to detect the presence of an antibody or antigen. Alternatively, a standard curve based on known concentration of an antibody or antigen can be prepared, from which the unknown concentration of a sample can be determined.

Detection is optical and accomplished by assessing the linked enzyme activity via incubation with a substrate to produce an optically measurable product. The most crucial element of the detection strategy is a highly specific antibody-antigen interaction. A detection enzyme or other tag can be linked directly to the primary antibody or introduced through a secondary antibody that recognises the primary antibody. It may be linked to a protein such as streptavidin if the primary antibody is biotin labelled. The most commonly used detection enzymes are horseradish peroxidase (HRP) and alkaline phosphatase (AP). Other enzymes have been used as well, but they have not gained widespread acceptance because of limited substrate options. The choice of substrate strongly depends upon the required assay sensitivity and the instrumentation available for signal-detection, such as spectrophotometer, fluorometer or luminometer.

Traditional ELISAs can be performed with a number of modifications to the basic procedure. The key step, immobilisation of an antigen of interest is accomplished by direct adsorption to the assay plate or indirectly via a capture antibody that has been immobilised on the plate. The antigen is then detected either directly (primary antibody is labelled) or indirectly (secondary antibody is labelled).

Among various standard assay formats, where differences in both capture and detection were the concern, it is important to differentiate between particular strategies that exist specifically for the detection step. Irrespective of the specific method by which an antigen is captured on the plate (either by direct adsorption to the surface, or through a pre-coated “capture” antibody, as in a sandwich format), it is the detection step (direct or indirect detection) that largely determines the sensitivity of the assay.

In-Cell ELISA is performed with cells that are plated and cultured overnight in standard microplates. After the cultured cells are fixed, permeabilised and blocked, target proteins are detected with antibodies. This is an indirect assay format, where secondary antibodies are either fluorescent (for direct measurement by a fluorescent plate reader or microscope) or enzyme-conjugated (for detection with a soluble substrate using a plate reader).

### Debye Length Limitation in Sorbent Assays

In most cases (in most biosensors or bioassays), molecular receptors bound to the transistor surface or to the plate surface are spatially separated from this surface by molecular cross-linkers or proteins of approximately 5-15 nm length. The molecule charges are therefore screened from the sensing surface by dissolved counter ions. As a result of the screening, the electro-static potential that arises from charges on the analyte molecule exponentially decreases to zero with increasing the distance from the sensing surface. This screening distance is defined as a “Debye length”, and it must be carefully selected when designing the receptor layer of any biosensor or sorbent assay in order to ensure the optimal sensing. For example, when detecting molecules in blood serum, the typical Debye screening length is 0.78 nm at temperature 36° C. with an electrical permittivity of 74.5 for water. This means that after this length, the Debye screening length is given by:

$${λ}_{D} = \sqrt{\frac{{ϵ}_{r} {ϵ}_{0} {k}_{b} T}{2 {n}_{0} {z}^{2} {e}^{2}}}$$

where n₀ is the bulk concentration of the electrolyte, ϵᵣ is the relative dielectric permittivity of the solvent (in case of water at 36° C. a value of 74.5), ϵ₀ is the permittivity of the vacuum, k_{b} is the Boltzmann constant, T is the temperature, z is the ion charge, and e is the elementary charge.

The screening length means that an electrical field originating from a point charge is dropped to its 1/e value (29%) in this length. Because of this limitation, charges from larger biomolecules (5-15 nm) cannot be detected in a serum sample. To overcome this problem, the charges should be attracted closer to the sensor or plate surface by using very short length receptors or by operating the sensor in completely desalted buffers for electronic molecular detection.

Currently, the Debye length limitation can be overcome by modification of the receptors and controlling the immobilisation density over the sensor sensing surface. Elnathan et al (in “Biorecognition Layer Engineering: Overcoming Screening Limitations of Nanowire-Based FET Devices”, Nanoletters 12, 2012, pp. 5245-5254) described this approach in detail and demonstrated the increased sensitivity of their sensor to troponin detection directly from serum for the diagnosis of acute myocardial infarction. However, the method proposed by them still requires receptor modification, which is a cumbersome biochemical procedure.

The present inventors have now unexpectedly found that performing a sorbent assay, such as In-Cell ELISA, on the sensor of the present invention, makes it possible to sense beyond the Debye screening length without modification of the receptors.

### SUMMARY OF THE INVENTION

The present application describes embodiments of a microelectronic device for sorbent, immunosorbent or cell sorbent assay, or flow cytometry, comprising:
(a) a plurality of microelectronic sensors, wherein said sensors are integrated into said device in rows and in columns, thereby forming an array, and each of said microelectronic sensors is connected to its dedicated electrical contact in a contact array;
(b) the contact array integrated within said microelectronic device;
(c) a row multiplexer connected to said contact array for addressing each and every sensor arranged in rows, selecting one of several analogue or digital input signals and forwarding the selected input into a single line;
(d) a column multiplexer connected to said contact array for addressing each and every sensor arranged in columns, selecting one of several analogue or digital input signals and forwarding the selected input into a single line; and
(e) an integrated circuit for storing and processing said signals;
characterised in that each of said microelectronic sensors comprises at least one open-gate pseudo-conductive high-electron mobility transistor (PC-HEMT), said transistor comprising:
   1) a multilayer hetero-junction structure made of gallium nitride (GaN) and aluminium gallium nitride (AlGaN) single-crystalline or polycrystalline semiconductor materials, and deposited on a substrate layer or placed on free-standing membranes, said structure comprising at least one buffer layer and at least one barrier layer, said layers being stacked alternately;
   2) a conducting channel comprising a two-dimensional electron gas (2DEG) or a two-dimensional hole gas (2DHG), formed at the interface between said buffer layer and said barrier layer, and upon applying a bias to said transistor, becoming capable of providing electron or hole current, respectively, in said transistor between source and drain contacts; and
   3) the source and drain contacts connected to said 2DEG or 2DHG conducting channel and to electrical metallisations for connecting said transistor to an electric circuit;
   said transistor is characterised in that the thickness of a top layer of said heterojunction structure in an open gate area of the transistor is 5-9 nanometres (nm) and the surface of said top layer has a roughness of 0.2 nm or less, wherein the combination of said thickness and said roughness of the top layer is suitable for creating a quantum electronic effect of operating said 2DEG or 2DHG channel simultaneously in both normally-on and normally-off operation modes of the channel, thereby making said transistor suitable for conducting electric current through said channel in a quantum well between normally-on and normally-off operation modes of the transistor.

In a particular embodiment, the microelectronic device of the present invention further comprises a Vivaldi antenna electrode or metamaterial electrode, such as Aharonov-Bohm antenna electrode, said Vivaldi antenna electrode or said metamaterial electrode being placed on the top layer between said source and drain contact in the open gate area of the transistor and capable of detecting electrical signals in the frequency range of 30 GHz to 300 THz.

In some embodiments, the PC-HEMT of each microelectronic sensor, in the open gate area, is not coated with a molecular or biomolecular layer and is capable of remotely detecting target (analyte) gases, chemical compounds or biomolecules from the environment. In other embodiments, the PC-HEMT further comprises at least one molecular or biomolecular layer immobilised within the open gate area of the transistor and capable of binding or adsorbing target (analyte) gases, chemical compounds or biomolecules from the environment. The molecular or biomolecular layer is selected, for example, from a cyclodextrin, 2,2,3,3-tetrafluoropropyloxy-substituted phthalocyanine or their derivatives. In certain embodiments, the molecular or biomolecular layer comprises capturing biological molecules, such as primary, secondary antibodies or fragments thereof against certain proteins to be detected, or their corresponding antigens, enzymes or their substrates, short peptides, specific DNA sequences, which are complimentary to the sequences of DNA to be detected, aptamers, receptor proteins or molecularly imprinted polymers.

In a particular embodiment, the PC-HEMT multilayer heterojunction structure comprises one of the following A-D structure:
A. (i) one top AlGaN layer recessed in an open gate area of the transistor to the thickness of 5-9 nm and having the surface roughness of 0.2 nm or less, and (ii) one bottom GaN buffer layer; said layers have Ga-face polarity, thus forming the two-dimensional electron gas (2DEG) conducting channel in said GaN layer, close to the interface with said AlGaN layer; or
B. (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have Ga-face polarity, thus forming a two-dimensional hole gas (2DHG) conducting channel in the top GaN layer, close to the interface with said AlGaN barrier layer; or
C. (i) one top GaN layer recessed in an open gate area of the transistor to the thickness of 5-9 nm and having the surface roughness of 0.2 nm or less, (ii) one bottom GaN buffer layer, and (iii) one AlGaN barrier layer in between; said layers have N-face polarity, thus forming a two-dimensional electron gas (2DEG) conducting channel in the top GaN layer, close to the interface with said AlGaN barrier layer; or
D. (i) one top AlGaN layer recessed in an open gate area of the transistor to the thickness of 5-9 nm and having the surface roughness of 0.2 nm or less, and (ii) one bottom GaN buffer layer; said layers have N-face polarity, thus forming a two-dimensional hole gas (2DHG) conducting channel in the GaN buffer layer, close to the interface with said AlGaN barrier layer.

The PC-HEMT source and drain contacts may be ohmic or non-ohmic. When the source and drain contacts are non-ohmic, the electrical metallisations of the transistor are capacitively-coupled to the 2DEG or 2DHG conducting channel for inducing displacement currents, thereby creating said non-ohmic source and drain contacts. In a particular embodiment, the transistor further comprises a dielectric layer deposited on top of said multilayer hetero-junction structure. In a specific embodiment, the thickness of the PC-HEMT top (barrier or buffer) layer in the open gate area is 6 to 7 nm, or 6.2 nm to 6.4 nm; and the surface of said top layer has a roughness of 0.2 nm or less, or 0.1 nm or less, or 0.05 nm or less. In another embodiment, the multilayer heterojunction structure further comprises a piezoelectric electro-optical crystal (EOC) transducer adapted to be brought into a contact with a medium to be sensed and adapted to be illuminated with a polarised light.

In a particular embodiment, the microelectronic device of the present invention is a microelectronic microwell plate (multiwell plate or microplate), and each of said microelectronic sensors is integrated at the bottom of its corresponding well of said microwell plate. In one embodiment, the microelectronic device of the present invention is a microelectronic microwell plate for sorbent, immunosorbent or cell sorbent assay, or flow cytometry, comprising:
(a) a plurality of the microelectronic sensors of the present invention, wherein each said sensor is integrated at the bottom of its corresponding well of said microwell plate and connected to its dedicated electrical contact in a contact array;
(b) the contact array integrated at the top of said microwell plate;
(c) a row multiplexer connected to said contact array for addressing each and every sensor arranged in rows, selecting one of several analogue or digital input signals and forwarding the selected input into a single line;
(d) a column multiplexer connected to said contact array for addressing each and every sensor arranged in columns, selecting one of several analogue or digital input signals and forwarding the selected input into a single line; and
(e) an integrated circuit for storing and processing said signals.

In a further embodiment, a method for sorbent, immunosorbent or cell sorbent assay of a sample containing a chemical compound or a biological compound to be tested in a gas phase or in a liquid phase comprises the following steps:
(1) Subjecting the sample to the microelectronic device of the present invention;
(2) Recording electrical signals received from said microelectronic device in a form of a source-drain electric current of the microelectronic sensor over time (I_{DS} dynamics);
(3) Transmitting the recorded signals from said microelectronic device to an external memory for further processing; and
(4) Converting the transmitted signals to digital signals and processing the digital signals in the external memory, comparing said I_{DS} dynamics with negative control chemical or biomolecular I_{DS} waveforms stored in the external memory, and extracting biochemical or biomolecular information from said waveforms in a form of readable data, thereby detecting and/or identifying a particular biological compound or cell in the sample and measuring their concentration or amount and biochemical or biophysical parameters.

Non-limiting examples of the chemical compound and biological compounds analysed by the method of the present invention are:
toxic metals, such as chromium, cadmium or lead,
regulated ozone-depleting chlorinated hydrocarbons,
food toxins, such as aflatoxin, and shellfish poisoning toxins, such as saxitoxin or microcystin,
neurotoxic compounds, such as methanol, manganese glutamate, nitrix oxide, tetanus toxin or tetrodotoxin, Botox, oxybenzone, Bisphenol A, or butylated hydroxyanisole,
explosives, such as picrates, nitrates, trinitro derivatives, such as 2,4,6-trinitrotoluene (TNT), 1,3,5-trinitro-1,3,5-triazinane (RDX), N-methyl-N-(2,4,6-trinitrophenyl)nitramide (nitramine or tetryl)trinitroglycerine, pentaerythritol tetranitrate (PETN), nitric ester, azide, derivates of chloric and perchloric acids, fulminate, acetylide, and nitrogen rich compounds, such as tetrazene, octahydro-1,3,5,7-tetranitro-1,3,5,7-tetrazocine (HMX), peroxide, such as triacetone trioxide, C4 plastic explosive and ozonidesor, or an associated compound of said explosives, such as a decomposition gases or taggants, and
biological pathogens, such as a respiratory viral or bacterial pathogen, an airborne pathogen, a plant pathogen, a pathogen from infected animals or a human viral pathogen.

In yet further embodiment, a method for in-vitro measurement of cell dynamics comprises the following steps:
(1) Subjecting a cell culture to a surface of the microelectronic sensor of the present invention or growing said cell culture directly on the surface of said sensor;
(2) Recording electrical signals received from said microelectronic sensor in a form of a source-drain electric current of the microelectronic sensor over time (I_{D}s dynamics) in real time;
(3) Transmitting the recorded electrical signals from said microelectronic sensor to an external memory for further processing; and
(4) Converting the transmitted signals to digital signals and processing the digital signals in the external memory, comparing said I_{DS} dynamics with negative control I_{DS} waveforms stored in the external memory, and extracting information on cell dynamics from said waveforms in a form of readable data.

Various embodiments may allow various benefits and may be used in conjunction with various applications. The details of one or more embodiments are set forth in the accompanying figures and the description below. Other features, objects and advantages of the described techniques will be apparent from the description and drawings and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Disclosed embodiments will be understood and appreciated more fully from the following detailed description taken in conjunction with the appended figures. The drawings included and described herein are schematic and are not limiting the scope of the disclosure. It is also noted that in the drawings, the size of some elements may be exaggerated and, therefore, not drawn to scale for illustrative purposes. The dimensions and the relative dimensions do not necessarily correspond to actual reductions to practice of the disclosure.

FIGS. 1a-1c schematically shows the quantum well at three different biasing conditions:

FIG. 1a: positive gate potential (+VG) is much higher than threshold voltage (VT),

FIG. 1b: 0V gate potential, and

FIG. 1c: negative gate potential (−VG) is below threshold voltage (VT).

FIGS. 2a-2b schematically shows a cross-sectional view (XZ) (a) and a top view (XY) (b) of the PC-HEMT of the present invention without a dielectric layer.

FIG. 2c schematically shows a cross-sectional view of the PC-HEMT of the present invention having non-ohmic (capacitively-coupled) contacts and no dielectric layer.

FIG. 2d schematically shows a cross-sectional view of the PC-HEMT of the present invention with highly-doped source and drain areas.

FIG. 2e schematically shows a cross-sectional view of the PC-HEMT of the present invention with a dielectric layer.

FIG. 2f schematically shows a cross-sectional view of the PC-HEMT of the present invention having non-ohmic (capacitively-coupled) contacts and a dielectric layer.

FIG. 2g schematically shows a cross-sectional view of the PC-HEMT of the present invention with free-standing membranes.

FIG. 2h illustrates a situation when the external pressure (mass effect) is applied on the sensor incorporating the PC-HEMT of FIG. 2g and transferred into a changed internal strain caused by bending.

FIG. 2i schematically shows a cross-sectional view of the PC-HEMT of the present invention with free-standing membranes and having non-ohmic (capacitively-coupled) contacts.

FIG. 3 schematically shows the dependence of the source-drain current (a charge carrier density) induced inside the 2DEG channel of a GaN/AlGaN HEMT on the thickness of the AlGaN layer recessed in the open gate area.

FIG. 4 illustrates a theory behind the 2DEG formation (charge neutrality combined with the lowest energy level) at the conduction band discontinuity.

FIG. 5a schematically shows the 2DEG area created in the step of the 2DEG-pattering via ion implantation during the manufacturing process. AZ 4533 is a positive thick resist.

FIG. 5b shows the lithographic mask of the sensor layout of the present invention.

FIG. 5c shows the lithographic image of the 2DEG channel formed with AZ 4533 thick resist lithography over the mask shown in FIG. 5b.

FIGS. 5d-5e show the mask and the corresponding lithographic image, respectively, of the sensor layout of the present invention.

FIG. 5f shows the ±2-μm alignment precision on 25×25 mm2 samples in the lithography of the sensor layout of the present invention.

FIG. 5g shows the lithographic images of the multichannel samples.

FIG. 5h shows the fixed sample on the Si—GaN/AlGaN wafer prepared for ion implantation and containing around 30-32 sensors with 4-8 channels on each sample.

FIG. 5i shows the lithographic image of the sensor layout with the AZ4533 resist after development, prepared for ion implantation.

FIG. 5j shows the 2DEG channels (dark) patterned by ion-implantation after the resist removal.

FIG. 5k shows the visible non-implanted area containing the conductive 2DEG channel.

FIG. 6a shows the AFM surface image of the top recessed layer of the PC-HEMT made by the manufacturing process of the present invention. The measured RMS value of the surface roughness is 0.674 nm in this case.

FIG. 6b shows the AFM surface image of the top recessed layer of the HEMT made by a conventional manufacturing process. The measured RMS value of the surface roughness is 1.211 nm in this case.

FIG. 6c shows the time-dependent plot of the drain-source electric current I_{DS} of the nitrogen oxide sensor of the present invention measuring 100 ppb of the NO₂ gas in humid air, where the sensor is based on the PC-HEMT made by the manufacturing process of the present invention.

FIG. 6d shows the time-dependent plot of the drain-source electric current I_{DS} of the nitrogen oxide sensor measuring 100 ppb of the NO₂ gas in humid air, where the sensor is based on the HEMT made by a conventional manufacturing process.

FIG. 7a schematically shows the formation of the 2DEG and 2DHG channels in the Ga-face three-layer Ga/AlGaN/GaN PC-HEMT structure.

FIG. 7b schematically shows the formation of the 2DEG and 2DHG channels in the N-face three-layer Ga/AlGaN/GaN PC-HEMT structure.

FIG. 8 schematically shows the formation of the 2DEG channel in the N-face three-layer GaN/AlGaN/GaN PC-HEMT structure with an ultrathin Al(GaN)N layer for improved confinement.

FIG. 9 illustrates the barrier layer/liquid or gas interface with the double layer formation, simplified equivalent interface circuitry and ion electrodynamics during exposure of the sensor to a charge (positive or negative).

FIGS. 10a-10b show the exemplary sensor layout of the present invention.

FIG. 11 shows the photograph of the exemplary sensor layout of the present invention having the layout shown in FIGS. 10a-10b.

FIG. 12a schematically shows the sensor circuit of the present invention.

FIGS. 12b-12c show different electronic configurations of the sensor of the present invention.

FIG. 13a shows the I_{DS} dynamics of the cardiac muscle cells (cardiomyocytes) on the surface of the sensor with the ultra-high signal to noise ratio.

FIG. 13b shows the expansion of the I_{DS} dynamics shown in FIG. 13a.

FIGS. 13c-13d shows the expansions of the I_{DS} dynamics shown in FIG. 13a in the narrower ranges showing the fine fingerprint of the beats.

FIGS. 14a-14d demonstrate the effect of noradrenaline (norepinephrine) and nifedipine on the I_{DS} dynamics of the cardiac muscle cells (cardiomyocytes) upon addition of these drugs to the cardiomyocytes medium together with the nutrient medium change:

FIGS. 14a-14c show the silencing of the cardiomyocytes upon addition of nifedipine at different time intervals.

FIG. 14d shows the recovering of the cell activity upon addition of noradrenaline.

FIGS. 15a-15b show the signal-to-noise ratio in the present experiment.

FIG. 16 shows the signal recorded by the sensor after cell death and removal from the surface of the sensor.

FIG. 17a schematically shows the microelectronic microwell plate of the present invention.

FIG. 17b shows the photographic image of the electronic 96-well plate of the present invention, in a bottom-up format.

FIGS. 17c and 17d show the photographic image of the electronic 96-well plate of the present invention, in a top-down format, and a schematic enlarged image of a single well from this plate, respectively.

FIG. 18 show the corrected plot averaged on four channels for the anti-pTau assay.

FIG. 19 shows the corrected plot averaged on four channels for the anti-testosterone hormone assay.

FIG. 20 shows the corrected plot averaged on six channels for the SCL70 antibody assay.

FIG. 21 shows the corrected plot averaged on ten channels for the EBV assay.

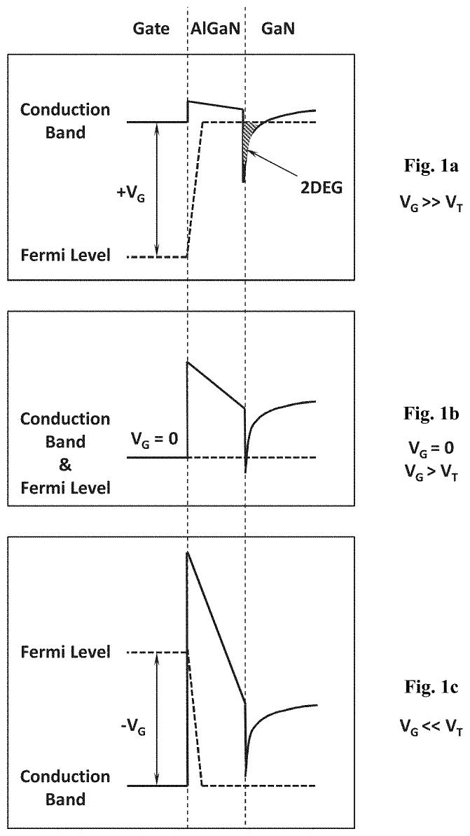

### DETAILED DESCRIPTION OF THE INVENTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising x and z” should not be limited to devices consisting only of components x and z. Also, the scope of the expression “a method comprising the steps x and z” should not be limited to methods consisting only of these steps.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached to”, “connected to”, “coupled with”, “contacting”, etc., another element, it can be directly on, attached to, connected to, coupled with or contacting the other element or intervening elements may also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “cell dynamics” includes any spatially and temporally regulated molecular events and processes occurring in biological cells. This includes but not limited to cellular behaviours such as cytokinesis, chemotaxis, cell division, cell differentiation, cell signalling, cell movement and cell contractility, transcriptional changes and changes in synaptic strength dependent upon spatially localised, temporally dynamic biochemical reactions. Tens of thousands of these events, such as changes in protein or lipid phosphorylation, localisation, and binding occur every second. The sensors of the present invention are capable of revealing how the cell and molecular dynamics lead to normal development or to diseases and help in understanding and preventing these diseases at the earliest stages and in the frame of the point-of-care diagnostics.

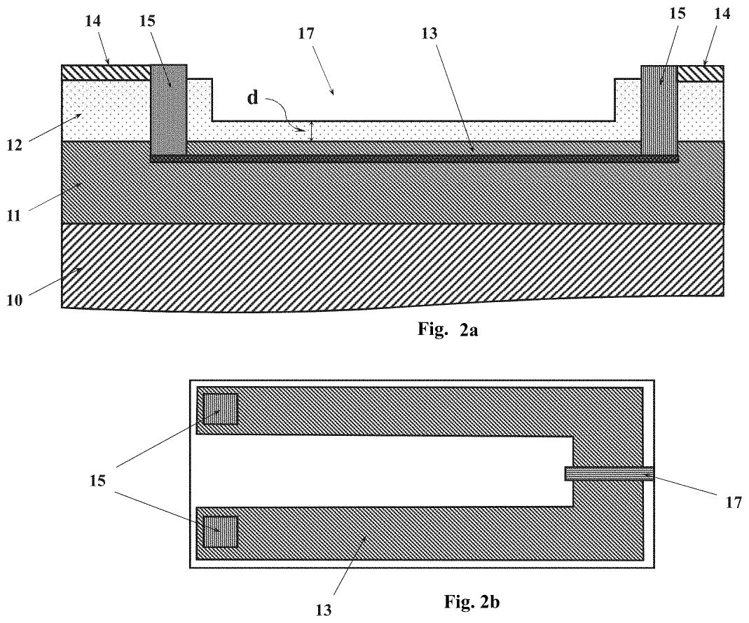

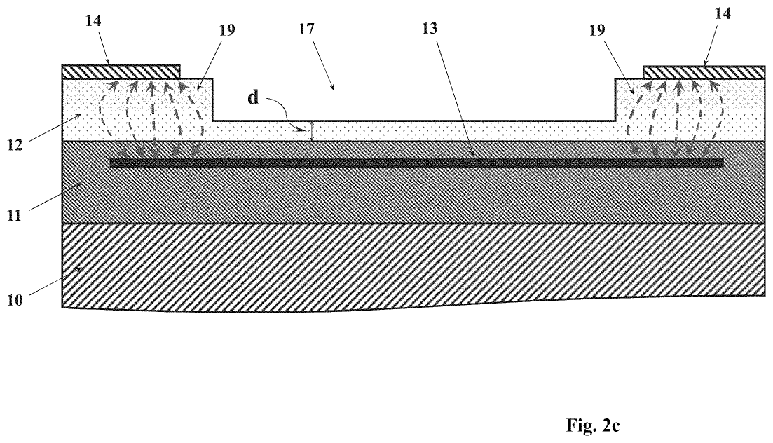

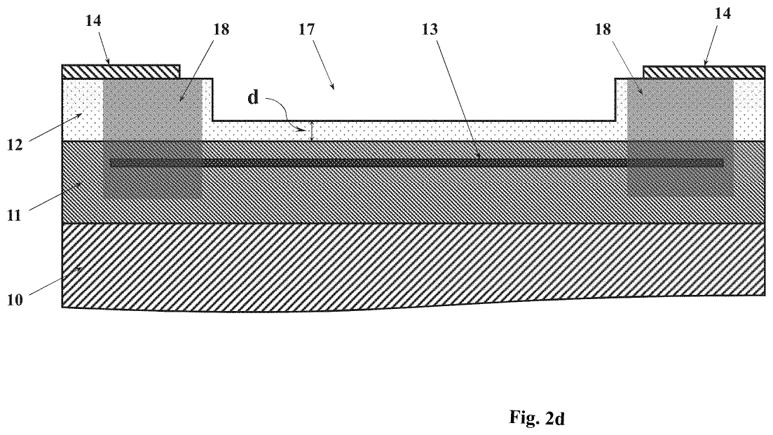

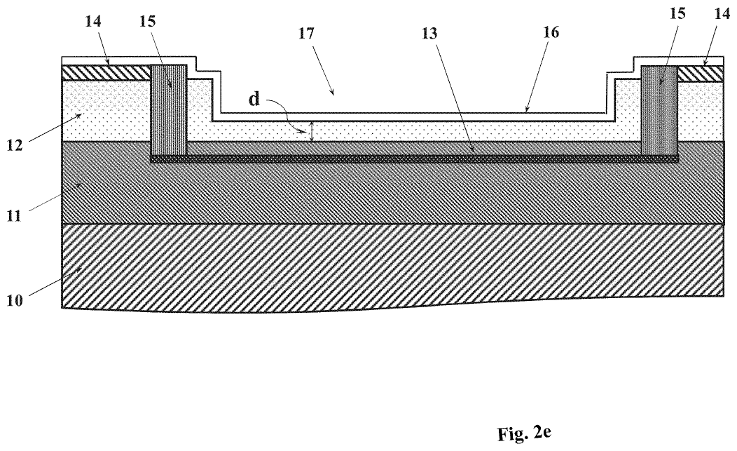

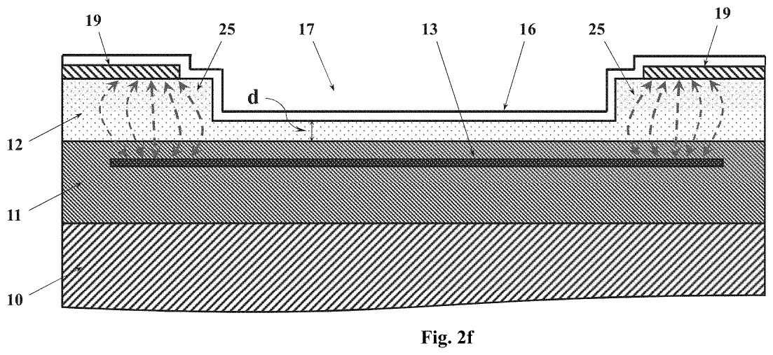

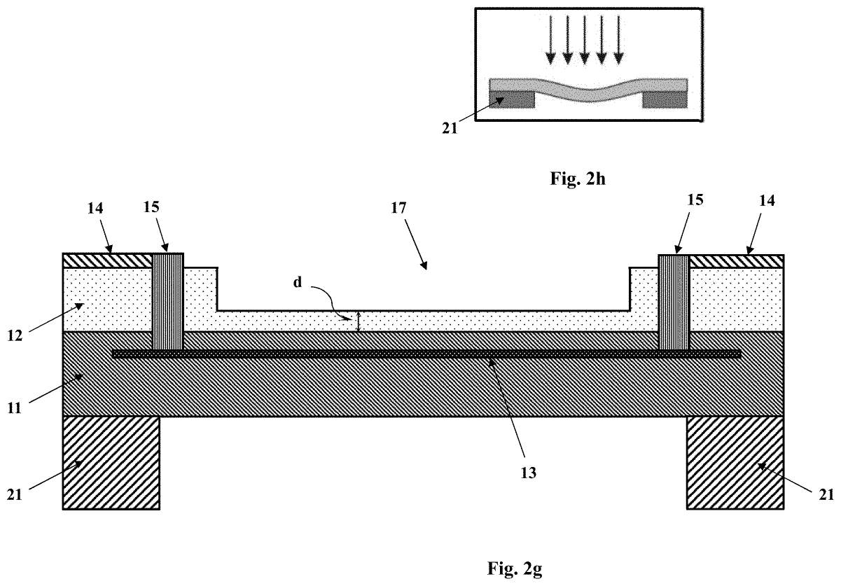

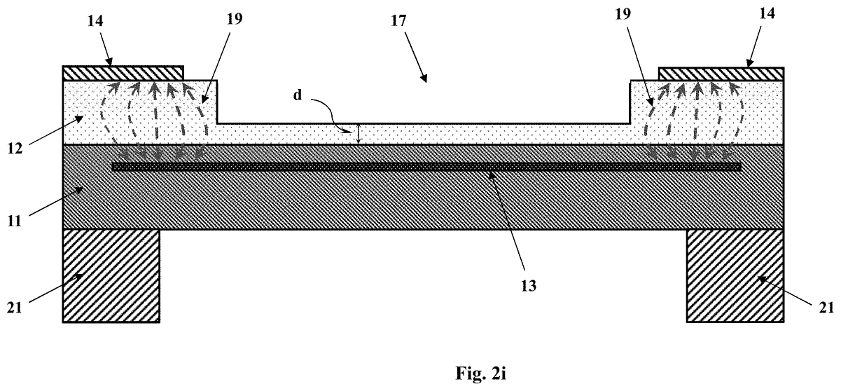

Reference is now made to FIGS. 2a-2i schematically showing the structure and topology of the PC-HEMT of the present invention, having different configurations. In one aspect, the present application describes an open-gate pseudo-conductive high-electron mobility transistor (PC-HEMT) suitable for use in sorbent, immunosorbent and sorbent cell assays and in the method for measuring cell dynamics, comprising:
(1) a multilayer heterojunction structure composed of III-V single-crystalline or poly-crystalline semiconductor materials, said structure comprising at least one buffer layer (11) and at least one barrier layer (12), said layers being stacked alternately, and said structure being deposited on a substrate layer (10) or placed on free-standing membranes (21);
(2) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between source and drain contacts;
(3) the source and drain contacts connected to said 2DEG or 2DHG channel (13) and to electrical metallisations (14) for connecting said transistor to an electric circuit; and
(4) an open gate area (17) between said source and drain contacts;
characterised in that the thickness (d) of a top layer of said structure in said open gate area is 5-9 nm which corresponds to the pseudo-conducting current range between normally-on and normally-off operation mode of the transistor, and the surface of said top layer has a roughness of about 0.2 nm or less.

The term “2DEG” mentioned in the present description and claims should not be understood or interpreted as being restricted to the two-dimensional electron gas. As stated above and will be explained later in this application, the two-dimensional hole gas may also be a possible current carrier in a specific heterojunction structure. Therefore, the term “2DEG” may be equally replaced with the term “2DHG” without reference to any particular PC-HEMT configuration.

The source and drain contacts connecting the PC-HEMT to the electric circuit may be ohmic or non-ohmic (capacitively-coupled, as will be described below). In one embodiment, FIGS. 2a-2b show a cross-sectional view (XZ) and a top view (XY) of the transistor of the present application, comprising:
a multilayer heterojunction structure composed of III-V single-crystalline or poly-crystalline semiconductor materials, said structure comprising at least one buffer layer (11) and at least one barrier layer (12), said layers being stacked alternately, and said structure being deposited on a substrate layer (10);
a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between source and drain contacts;
source and drain ohmic contacts (15) connected to said 2DEG conducting channel (13) and to electrical metallisations (14) for connecting said transistor to an electric circuit; and
an open gate area (17) between said source and drain ohmic contacts (15);
characterised in that the thickness (d) of a top layer of said structure in said open gate area is 5-9 nm which corresponds to the pseudo-conducting current range between normally-on and normally-off operation mode of the transistor, and the surface of said top layer has a roughness of about 0.2 nm or less.

Further, FIG. 2c shows a cross-sectional view of the PC-HEMT of another embodiment comprising:
1) a multilayer heterojunction structure composed of III-V single-crystalline or poly-crystalline semiconductor materials, said structure comprising at least one buffer layer (11) and at least one barrier layer (12), said layers being stacked alternately, and said structure being deposited on a substrate layer (10);
2) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between non-ohmic source and drain contacts;
3) electrical metallisations (14) capacitively-coupled to said 2DEG channel (13) for inducing displacement currents (19), thereby creating non-ohmic source and drain contacts connecting said transistor to an electric circuit; and
4) an open gate area (17) between said source and drain non-ohmic contacts;
characterised in that the thickness (d) of a top layer of said structure in said open gate area is 5-9 nm which corresponds to the pseudo-conducting current range between normally-on and normally-off operation mode of the transistor, and the surface of said top layer has a roughness of about 0.2 nm or less.

“Capacitive coupling” is defined as an energy transfer within the same electric circuit or between different electric circuits by means of displacement currents induced by existing electric fields between circuit/s nodes. In general, ohmic contacts are the contacts that follow Ohm's law, meaning that the current flowing through them is directly proportional to the voltage. Non-ohmic contacts however do not follow the same linear relationship of the Ohm's law. In other words, electric current passing through non-ohmic contacts is not linearly proportional to voltage. Instead, it gives a steep curve with an increasing gradient, since the resistance in that case increases as the electric current increases, resulting in increase of the voltage across non-ohmic contacts. This is because electrons carry more energy, and when they collide with atoms in the conducting channel, they transfer more energy creating new high-energy vibrational states, thereby increasing resistance and temperature.

When electrical metallisations are placed over single-crystalline or polycrystalline semiconductor material, the “Schottky contact” or “Schottky barrier contact” between the metal and the semiconductor occurs. Energy of this contact is covered by the Schottky-Mott rule, which predicts the energy barrier between a metal and a semiconductor to be proportional to the difference of the metal-vacuum work function and the semiconductor-vacuum electron affinity. However, this is an ideal theoretical behaviour, while in reality most interfaces between a metal and a semiconductor follow this rule only to some degree. The boundary of a semiconductor crystal abrupt by a metal creates new electron states within its band gap. These new electron states induced by a metal and their occupation push the centre of the band gap to the Fermi level. This phenomenon of shifting the centre of the band gap to the Fermi level as a result of a metal-semiconductor contact is defined as “Fermi level pinning”, which differs from one semiconductor to another. If the Fermi level is energetically far from the band edge, the Schottky contact would preferably be formed. However, if the Fermi level is close to the band edge, an ohmic contact would preferably be formed. The Schottky barrier contact is a rectifying non-ohmic contact, which in reality is almost independent of the semi-conductor or metal work functions.

Thus, a non-ohmic contact allows electric current to flow only in one direction with a non-linear current-voltage curve that looks like that of a diode. On the contrary, an ohmic contact allows electric current to flow in both directions roughly equally within normal device operation range, with an almost linear current-voltage relationship that comes close to that of a resistor (hence, “ohmic”).

Reference is now made to FIG. 2c illustrating the situation when an electrical connection of the transistor to the 2DEG channel is realised via capacitive coupling to electrical metallisations through a Schottky barrier contact. This coupling becomes possible only if sufficiently high AC frequency, higher than 30 kHz, is applied to the metallisations. The electrical metallisations capacitively coupled to the 2DEG channel utilise the known phenomenon of energy transfer by displacement currents. These displacement currents are induced by existing electrical fields between the electrical metallisations and the 2DEG conducting channel operated in the AC frequency mode through the Schottky contact as explained above.

FIG. 2d schematically shows a cross-sectional view of the PC-HEMT of an embodiment of the present application with highly-doped source and drain areas (18). In that case, the strong doping of the source and drain areas may result in a band-edge mismatch. However, if the semiconductor is doped strongly enough, it will form a certain potential barrier, low enough for conducting electrons to have a high probability of tunneling through this barrier, and therefore conducting an electric current through the 2DEG channel.

An electrical connection to the 2DEG channel shown in FIG. 2d is realised with highly doped semiconductor areas (18) overlapping the 2DEG channel and having a very low electrical resistance. Dopant ions such as boron (B″), phosphorus (P′) or arsenic (Ask) are generally created from a gas source, so that the purity of the source can be very high. When implanted in a semiconductor, each dopant atom creates a charge carrier in the semiconductor material after annealing. Holes are created for a p-type dopant, and electrons are created for an n-type dopant, modifying conductivity of the semiconductor in its vicinity. As⁺ can be used for n-type doping, while B⁺ and P⁺ ions can be used for p-type doping. For example, in case of the AlGaN/GaN structure, the source and drain areas of the silicon structure are heavily doped with either B⁺ or P⁺ to create an electrical connection to the 2DEG channel. The silicon layers have a very low electrical junction resistance between each other in that case, and in order to induce an electrical current in the 2DEG channel, the metallisations are placed on top of the source and drain areas and connected to a circuit.

The third option would be the use of the photo effect that may also induce an electric current in the 2DEG channel. In order to couple the light excitation with the electronic effects in the conductive 2DEG channel, a photo effect in a silicon layer should be created. Regarding the direct photo effect, it is well known that light can only be absorbed when the energy of the absorbed photon (E=hv) is large enough for an electron to be excited into the valence band. In that case, E is the photon energy, h is Planck's constant and v is the frequency of the photon. The frequency is coupled to the wavelength λ of light by the constant speed of light c=λv. Typically, the bandgap of silicon at room temperature is 1.12 eV, which means that silicon becomes transparent for wavelength larger than 1240 nm, which is the near infrared range.

For smaller wavelength (i.e. larger energy of the photons), electron/hole pairs are generated leading to a photocurrent. In the fully-depleted, intrinsically doped silicon structures, this results in a higher charge carrier density and consequently, higher sensitivity. For these structures, light is adsorbed in the whole visible range making such devices ideal photodetectors. The mechanism that allows the silicon semiconductor to become photosensitive to irradiation with light has already been described in literature. In the direct photo effect, it can be tuned by the size, crystalline direction and surface termination. These effects actually originate from two-dimensional quantum confinement of electrons in the nano-sized 2DEG structure.

Although irradiation of the silicon structure with light of larger wavelengths with photon energies below the bandgap does not have enough energy to excite carriers from the valence to the conduction band in bulk silicon, the electron/hole pairs can also be generated between the valence band and surface states, and the donor-like surface trap states can still be formed (see the definition and explanation of the surface trap states below). The electrons actually deplete these holes trapped at the surface and hence, modulate the gate field. The photogenerated holes are confined to the centre of the silicon structure by the gate field, where they increase the conduction of the 2DEG channel, because of the band bending. The holes increase the channel conductivity for a certain lifetime until they are trapped (recaptured) at the surface. The gain of the transistor can be extremely huge if this re-trapping lifetime is much longer than the holes transit time.

If the source and drain contacts are non-ohmic (capacitively-coupled), in order to electrically contact the 2DEG channel underneath, which is about 7-20 nm bellow metallisations (14), the AC frequency regime is used. The capacitive coupling of the non-ohmic metal contacts with the 2DEG channel is normally induced at the frequency higher than 30 kHz. In the case of the non-ohmic contacts, the DC readout cannot be performed. Instead, the AC readout or impedance measurements of the electric current flowing through the 2DEG channel are carried out.

Thus, the significant features of the PC-HEMT structure are that:
(a) the thickness of the top barrier layer in the open gate area is 5-9 nm, preferably 6-7 nm, more preferably 6.3 nm, which corresponds to the pseudo-conducting current range between normally-on and normally-off operation mode of the transistor, and
(b) the surface of the top barrier layer has a roughness of 0.2 nm or less, preferably 0.1 nm or less, more preferably 0.05 nm.

The same transistors of the embodiments shown in FIGS. 2a-2c, but further comprising an optional dielectric layer (16), which is deposited on top of the barrier layer (12) of the transistors, are schematically shown in FIGS. 2e and 2f, respectively. The optional dielectric layer (16), which is used for device passivation, is made for example of SiO—SiN—SiO (“ONO”) stack having thickness of 100-100-100 nm or SiN—SiO—SiN (“NON”) stack having the same thicknesses. This dielectric layer (16) is deposited on top of the barrier layer by a method of plasma-enhanced chemical vapour deposition (PECVD), which is a stress-free deposition technique.

FIG. 2g shows a cross-sectional view of the PC-HEMT configuration of an embodiment with free-standing membranes, comprising:
1) a multilayer heterojunction structure composed of III-V single-crystalline or poly-crystalline semiconductor materials, said structure comprising at least one buffer layer (11) and at least one barrier layer (12), said layers being stacked alternately, and said structure being placed on free-standing membranes (21);
2) a conducting channel (13) comprising a two-dimensional electron gas (2DEG) or a two-dimensional hole gas (2DHG), formed at the interface between said buffer layer (11) and said barrier layer (12), and upon applying a bias to said transistor, capable of providing electron or hole current, respectively, in said transistor between source and drain contacts;
3) source and drain ohmic contacts (15) connected to said 2DEG conducting channel (13) and to electrical metallisations (14) for connecting said transistor to an electric circuit; and 4) an open gate area (17) between said source and drain ohmic contacts (15);
characterised in that the thickness (d) of a top layer of said structure in said open gate area is 5-9 nm which corresponds to the pseudo-conducting current range between normally-on and normally-off operation mode of the transistor, and the surface of said top layer has a roughness of about 0.2 nm or less.

The PC-HEMT shown in FIG. 2g, placed on free standing membranes may be used in piezoelectric or “pressure-sensitive” sensors of the present invention. These sensors are capable of measuring very small pressures, for example when molecules or cells from a sample contact the surface of the transistor. These sensors use the free-standing membranes for creating a mass-loading effect which makes it possible to increase selectivity of the sensors via adding mechanical stress (mass-loading effect) as an additional parameter of the PC-HEMT-based sensor. The free-standing membranes (21) are very flexible free-standing columns of substrate composed of sapphire, silicon, silicon carbide, gallium nitride or aluminium nitride, preferably gallium nitride, having thickness of 0.5-2 μm. The free-standing substrate membranes are very sensitive to any tensile, compressive or mechanical stress changes on the surface of the multilayer hetero-junction structure. This results in a mass loading effect, which will be discussed below.

In general, mechanical sensors, much like pressure sensors, are based on the measurement of the externally induced strain in the heterostructures. The pyroelectric properties of group-III-nitrides, such as gallium nitride (GaN), allow two mechanisms for strain transduction: piezoelectric and piezoresistive. The direct piezoelectric effect is used for dynamical pressure sensing. For measurements of static pressure, such sensors are not suitable due to some leakage of electric charges under the constant conditions. For static operation, the piezoresistive transduction is more preferable.

Piezoresistive sensors using wide band gap materials have been previously employed using hexagonal silicon carbide bulk materials for high temperature operation. The piezoresistivity of GaN and AlGaN structures was found to be comparable to silicon carbide. However, piezoresistivity can be further amplified by HEMT structure, as taught by Martin Eickhoff et al in “Piezoresistivity of AlₓGa₁₋ₓN layers and AlₓGa₁₋ₓN/GaN heterostructures”, Journal of Applied Physics 90, 2001, 3383.

For piezoresistive strain sensing at relatively lower pressures (or pressure differences), diaphragm or membranes should be used, where the external pressure is transferred into a changed internal strain caused by bending, as shown in FIG. 2h. The resulting change in polarization alters the 2DEG channel current which is measured.

Eickhoff et al (2001) conducted the first experiments on AlGaN/GaN hetero-structures where the 2DEG channel confined between the upper GaN and AlGaN barrier layer and demonstrated the linear dependence of the 2DEG channel resistivity on the applied strain. Moreover, a direct comparison to cubic SiC and a single AlGaN layer clearly demonstrated the superior piezoresistive properties of the latter. From these results, it is clear that the interaction of piezoelectric and piezoresistive properties improves the sensitivity of pressure sensors by using GaN/AlGaN heterostructures confined with the 2DEG channel.

The sensor configuration shown in FIGS. 2g and 2i involves piezoelectrically coupled, charge and mass sensitive, free-standing GaN membranes, which are prepared, for example, according to U.S. Pat. No. 8,313,968, and offer an elegant and effective solution to achieve both downscaling and an integrated all-electrical low-power sensing-actuation. As mentioned above, GaN exhibits both, piezo- and pyro-electrical properties, which can be functionally combined. Whereas the piezoelectricity enables realisation of an integrated coupling mechanism, the 2DEG additionally delivers a pronounced sensitivity to mechanical stress and charge, which allows the sensor to use the pyroelectric effects. The dynamic change in 2DEG conductivity is also caused by a change in piezoelectric polarisation.

The electrical metallisations (14) connect the PC-HEMT to an electric circuit and allow electric current to flow between the source and drain contacts. The electrical metallisations (14) are made of metal stacks, such as Cr/Au, Ti/Au, Ti/W, Cr/Al and Ti/Al. The Cr or Ti layers of the metal stack is, for example, of 5-10 nm thickness, while the second metal layer, such as Au, W and Al, is of 100-400 nm thickness. The actual metallisations (14) are chosen according to the established technology and assembly line at a particular clean room fabrication facility. The source and drain ohmic contacts are usually made of metal stacks, such as Ti/Al/Mo/Au, Ti/Al/Ni/Au, Ti/Au and Ti/W having the thickness of 15-50 nm. The non-ohmic contacts on the other hand are capacitively coupled to the conducting 2DEG channel (13) via displacement currents (19).

In yet further embodiment, substrate layer (10) comprises a suitable material for forming the barrier layer and is composed, for example, of sapphire, silicon, silicon carbide, gallium nitride or aluminium nitride. The hetero-junction structure (11, 12) is deposited on the substrate layer (10), for example, by a method of metalorganic chemical vapour deposition (MOCVD), and it forms a two-dimensional electron gas (2DEG) channel (13) in the close proximity to the interface between the buffer layer (11) and the top barrier layer (12). The top barrier layer (12) then may be either recessed or grown as a thin layer between the source and drain contacts, thereby forming an open gate area.

The 2DEG/2DHG channel (13) formed near the interface between the buffer layer (11) and the barrier layer (12) serves as a main sensitive element of the transistor reacting to a surface charge and potential. The 2DEG/2DHG channel (13) is configured to interact with very small variations in surface or proximal charge or changes of electrical field on the barrier layer/liquid-air or barrier layer/metal/liquid-air interfaces interacting with the donor-like surface trap states of the barrier layer. This will be defined and discussed below in detail.

“Open gate area” of the PC-HEMT is defined as an area between the source and drain contacts of the transistor which is directly exposed to a conductive medium, such as liquid or gas capable of conducting current. An example of the conductive liquid is an electrolyte saline solution. In this case, instead of the fixed gate voltage, which is normally applied to a gate electrode, a reference potential is applied to the electrolyte-semiconductor system, via an optional reference electrode that is dipped into the electrolyte. As a result, in the absence of the physical gate, the electrolyte itself becomes an open gate of the transistor. This will be explained in more detail below.

The specific thickness of the top barrier layer (12) in the open gate area is achieved by either dry etching the semiconductor material of the barrier layer (12), i.e. recessing the layer in the open gate area with the etching rate of 1 nm per 1-2 min in a controllable process, or coating the buffer layer (11) in the open gate area with an ultrathin layer of the III-V semiconductor material. In order to increase the charge sensitivity of the transistor, the surface of the recessed ultrathin barrier layer is post-treated with plasma (chloride) epi-etch process. Consequently, the natively passivated surface is activated by the plasma etch to create an uncompensated (ionised) surface energy bonds or states, which are neutralized after MOCVD growing.

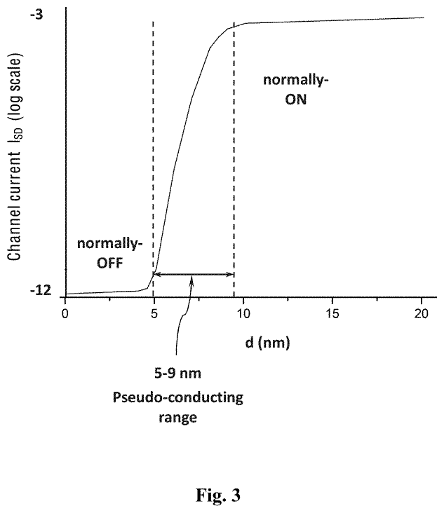

FIG. 3 shows the dependence of the source-drain current (a charge carrier density) on the barrier layer thickness recessed in the open gate area. As seen from the plot, the HEMTs that have a thickness of the barrier layer in the open gate area larger than about 9 nm are normally-on devices. In such devices, due to the inherent polarisation effects present in the III-V materials, a thin sheet of charges is induced at the top and bottom of the interfaces of the barrier layer. As a result, a high electric field is induced in the barrier layer, and surface donor states at the top interface start donating electrons to form the 2DEG channel at the proximity of the hetero-junction interface without the application of a gate bias. These HEMTs are therefore normally-on devices. On the other hand, the HEMTs that have a thickness of the barrier layer in the open gate area lower than about 5 nm act as normally-off devices.

The top barrier layer recessed or grown in the open gate area to 5-9 nm is optimised by minimising the roughness of the top semiconductor layer to 0.2 nm and less. The resulted structure was surprisingly found to significantly enhance sensitivity of the sensor. This specific thickness of 5-9 nm of the top barrier layer in the open gate area with the roughness of 0.2 nm or less corresponds to the “pseudo-conducting” current range between normally-on and normally-off operation modes of the transistor and requires further explanation.

“Pseudo-conducting” current range of the HEMT is defined as an operation range of the HEMT between its normally-on and normally-off operation modes. “Trap states” are states in the band-gap of a semiconductor which trap a carrier until it recombines. “Surface states” are states caused by surface reconstruction of the local crystal due to surface tension caused by some crystal defects, dislocations, or the presence of impurities. Such surface reconstruction often creates “surface trap states” corresponding to a surface recombination velocity. Classification of the surface trap states depends on the relative position of their energy level inside the band gap. The surface trap states with energy above the Fermi level are acceptor-like, attaining negative charge when occupied. However, the surface trap states with energy below the Fermi level are donor-like, positively charged when empty and neutral when occupied. These donor-like surface trap states are considered to be the source of electrons in the formation of the 2DEG channel. They may possess a wide distribution of ionization energies within the band gap and are caused by redox reactions, dangling bonds and vacancies in the surface layer. A balance always exists between the 2DEG channel density and the number of ionised surface donors which is governed by charge neutrality and continuity of the electric field at the interfaces.

Thus, the donor-like surface traps formed at the surface of the barrier layer of the HEMT are one of the most important sources of the 2DEG in the channel. However, this only applies for a specific barrier layer thickness. In a relatively thin top barrier layer, the surface trap state is below the Fermi level. However, as the top barrier layer thickness increases, the energy of the surface trap state approaches the Fermi energy until it coincides with it. The thickness of the top barrier layer corresponding to such situation is defined as “critical”. At this point, electrons filling the surface trap state become pulled to the channel by the strong polarisation-induced electric field found in the barrier to form the 2DEG instantly.

If the surface trap states are completely depleted, further increase in the barrier layer thickness will not increase the 2DEG density. Actually, if the 2DEG channel layer fails to stretch the barrier layer, the later will simply relax. Upon relaxation of the barrier layer, crystal defects are created at the interface between the buffer layer and the barrier layer, and the piezoelectric polarisation instantly disappears causing deterioration in the 2DEG density.

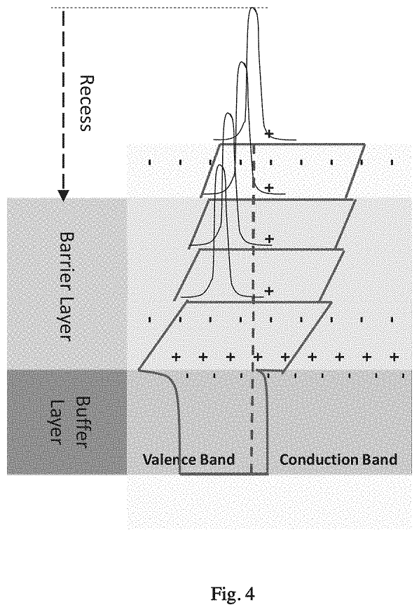

In order to illustrate the above phenomenon of pseudo-conducting current, reference is now made to the following figures. As mentioned above, FIG. 3 shows the dependence of the source-drain current (a charge carrier density) on the recessed AlGaN barrier layer thickness. An energy equilibrium between the donor surface trap states and the AlGaN tunnel barrier leads to formation of the 2DEG (charge neutrality combined with the lowest energy level) at the conduction band discontinuity. As explained above, decrease in the thickness of the top barrier layer results in increase of the energy barrier. As a result, the ionisable donor-like surface trap states, which are responsible for electron tunneling from the surface to 2DEG, drift bellow the Fermi level, thereby minimizing the electron supply to the 2DEG channel. This theoretical situation is schematically illustrated in FIG. 4. Therefore, the recess of the top AlGaN layer from 9 nm to 5 nm leads to extremely huge drop in the 2DEG conductivity for six orders of magnitude.

In view of the above, it is clear that the mechanism of the 2DEG depletion based on recessing the top barrier layer is strongly dependent on the donor-like surface trap states (or total surface charge). As the thickness of the barrier layer decreases, less additional external charge is needed to apply to the barrier layer surface in order to deplete the 2DEG channel. There is a critical (smallest) barrier thickness, when the 2DEG channel is mostly depleted but still highly conductive due to a combination of the energy barrier and the donor surface trap states energy. At this critical thickness, even the smallest energy shift at the surface via any external influence, such as surface reaction, charging etc., leads immediately to very strong 2DEG depletion. As a result, the surface of the top barrier layer at this critical thickness is extremely sensitive to any smallest change in the electrical field of the surroundings.

Thus, it has been found that the recess of the top layer in the open gate area from 9 nm down to 5 nm drastically reduces the 2DEG density, brings the transistor to the “near threshold” operation and results in highly increased surface charge sensitivity. The specific 5-9 nm thickness of the transistor's top layer responsible for its surprising pseudo-conducting behaviour gives the transistor the incredible sensitivity. So, when it comes into a contact with an ionic fluid or body skin, it opens up the gate to be able to do the ultrasensitive sensing. This thickness must be optimised for significantly enhancing sensitivity of the sensor. This specific thickness of the top layer was surprisingly found to correspond to the “pseudo-conducting” current range between normally-on and normally-off operation modes of the 2DEG channel and requires further explanation.

The top layer is recessed to this specific thickness after subjecting to short plasma activation by an ultra-low damage reactive-ion etching technique using inductively-coupled plasma (ICP) with a narrow plasma-ion energy distribution. Such short plasma treatment allows much lower roughness of the surface, which is a function of the semiconductor vertical damage depth during the plasma etching process. Such low surface roughness (about 0.2 nm and less) can be achieved only via this ICP-RIE ultra low damage etching process with a narrow plasma-ion energy distribution, and this inherently results in a very low vertical damage depth to the top layer, which allows the minimal surface scattering and minimal surface states-2DEG channel interaction with the maximum signal-to-noise ratio of the sensor. Thus, the depth effect of the vertical sub-nanometre damage to the top recessed layer, due to an ultra-low damage ICP-RIE etching process with a very narrow plasma-ion energy distribution, is the only way to optimally achieve the required sub-nanometre roughness of the semiconductor surface. This inherently results in an adjustable pseudo-conductive working point with the highest charge sensitivity ever possible. This depth effect is always inherent to the sub-nanometre roughness of the semiconductor surface, which was measured using AFM (atomic force microscope).

Thus, in addition to the recessed top layer thickness, roughness of the top layer surface is another very important parameter that has not been previously disclosed. It has been surprisingly found that the roughness of the top layer surface (in the open gate sensitive area) bellow 0.2 nm prevents scattering of the donor-like surface trap states. Thus, combination of these two features: 5-9 nm thickness of the top layer in the open gate area and strongly reduced roughness of its surface (bellow 0.2 nm) make the sensor incredibly sensitive.

In a certain aspect, the method for manufacturing of the PC-HEMTs of the present invention comprises the following steps:
Step 1: Plasma-enhanced atomic layer deposition (ALD) of alumina (Al₂O₃) on a pre-aligned masked Si—GaN/AlGaN wafer with nitrogen-plasma de-trapping for the thickness of the Al₂O₃ layer being 3-10 nm. The Al₂O₃ layer thickness was measured with an X-ray reflectometer.
Step 2: Plasma-enhanced atomic layer deposition (ALD) pattering of the wafer coated with the thin Al₂O₃ layer in Step 1, with hydrogen fluoride (HF) or using the aforementioned reactive-ion etching (ME) technique.
Step 3: Optionally creating the source and drain ohmic contacts (in case ohmic contacts are required) on the coated wafer obtained in Step 2 from metal stacks, for example Ti/Al/Mo/Au, Ti/Al/Ni/Au, Ti/Au and Ti/W, having 15-50 nm thickness, using spin-coating technique or e-beam physical vapour deposition (VPD) of the stack metals. The deposition rates using the e-VPD technique were determined for the ohmic-stack metals using the Dektak Profilometer with dummy lift-off samples.
Step 4: Two-dimensional electron gas (2DEG) channel-pattering of the wafer obtained in Step 3 with argon- or nitrogen-ion implantation.
Step 5: Plasma-enhanced chemical vapour deposition (CVD) of the ONO stack over the wafer obtained in Step 4. This is the stress-free technique to deposit the layer of the SiO—SiN—SiO stack having an exemplary thickness of about 200-300 nm and structured by the ICP-RIE dry etching, which is the CF4-based etching method. In this step, the pseudo-conducting channel areas and ohmic electrical contact pads of the transistor become available.
Step 6: Optional lift-off deposition of an Au or Ti/W-CMOS-gate electrode (in case a gate electrode is to be deposited on the top layer of the heterojunction structure for an integrated MMIC-HEMT-based amplifier manufacturing).
Step 7: Optional plasma-enhanced ALD pattering with RIE or HF above sensing area (in case the plasma-enhanced ALD layer deposited in Step 1 is removed separately to ONO stack).
Step 8: Atomic layer etching (ALE) of the wafer obtained in Steps 5-7. This sophisticated technique carried out in the clean manufacturing cluster of the applicant is the only technique allowing the removal of individual atomic layers (the top atomic layers of the wafer). ALE is a way better-controlled technique than RIE, though it has not been commercially used until now because very sophisticated gas handling is required, and removal rates of one atomic layer per second are the real state of the art. This step is the step of creating the pseudo-conducting working point of the transistor, because ALE allows achieving the specific thickness of 5-9 nm thickness of the top layer in the open gate area with the extremely low surface roughness of the top layer below 0.2 nm.
Step 9: Optional plasma-enhanced CVD or ALD of the dielectric layer used for device passivation and in some gas sensors.
Step 10: Optional deep reactive-ion etching (DRIE or Bosch process) of the Si-substrate under sensing areas (in case the substrate is on the free-standing membranes—used, for example, in RF-HEMTs, FBAR and SAW sensors).

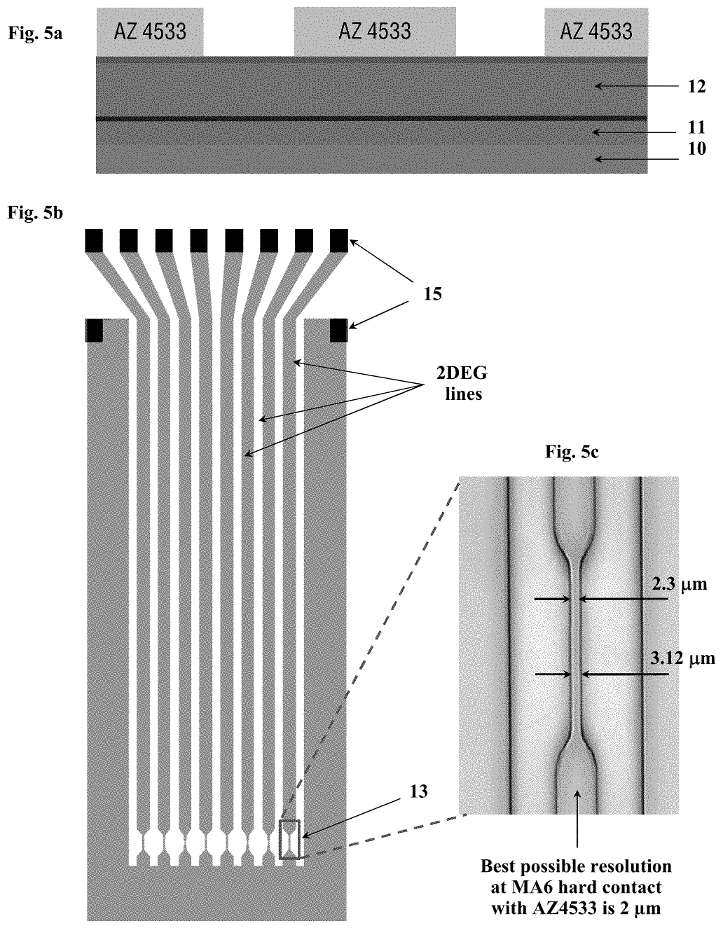

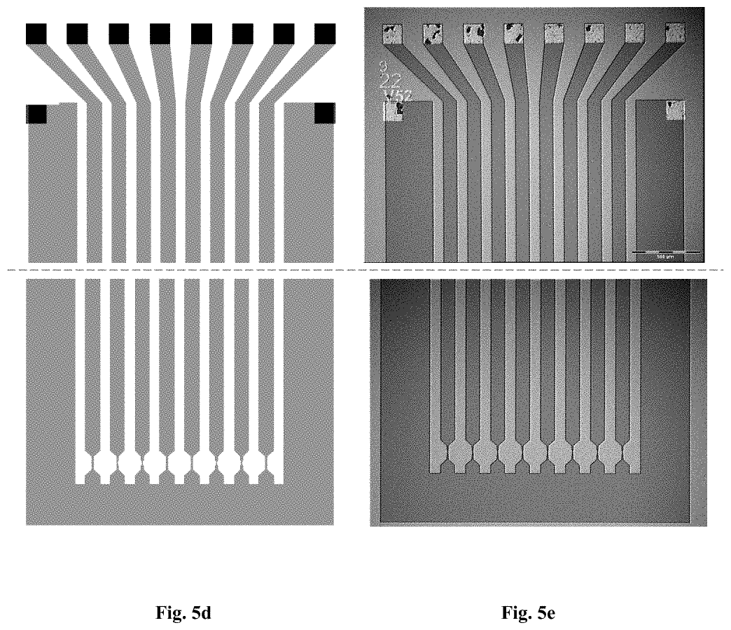

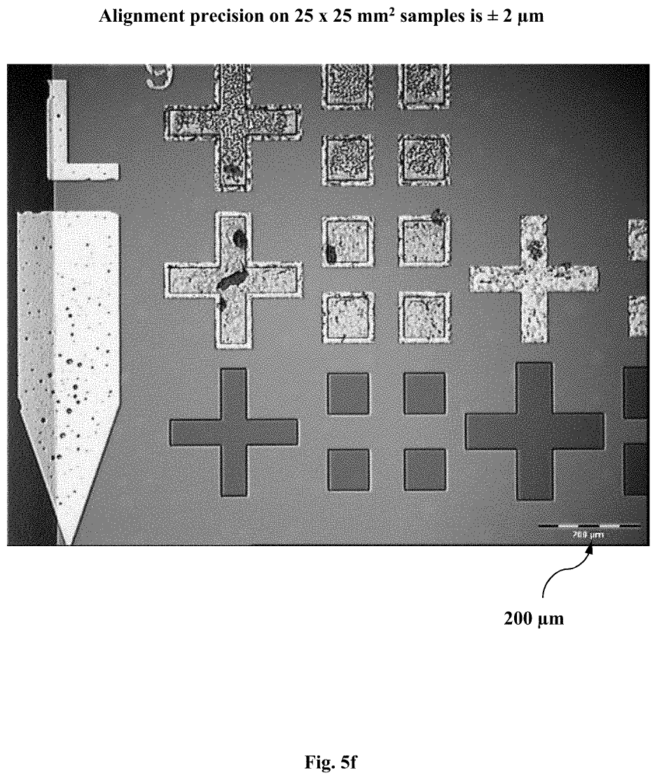

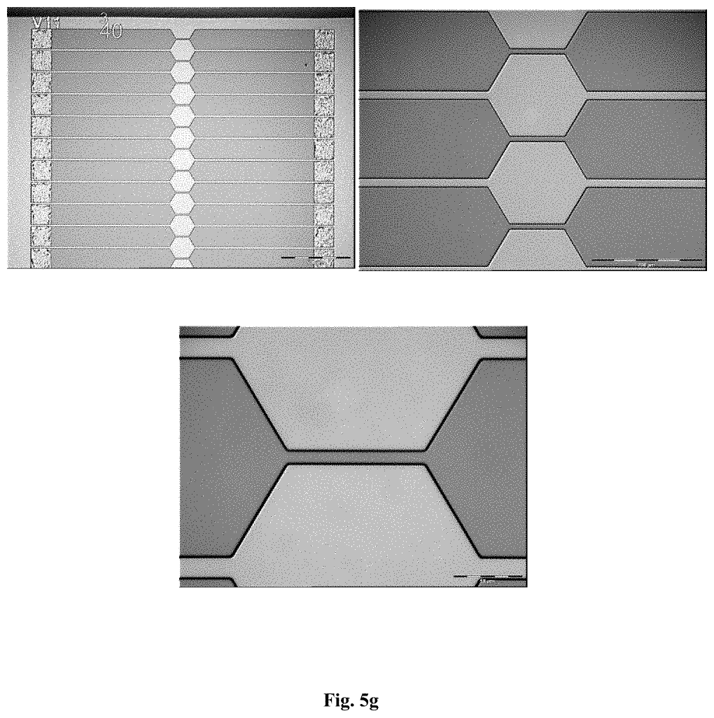

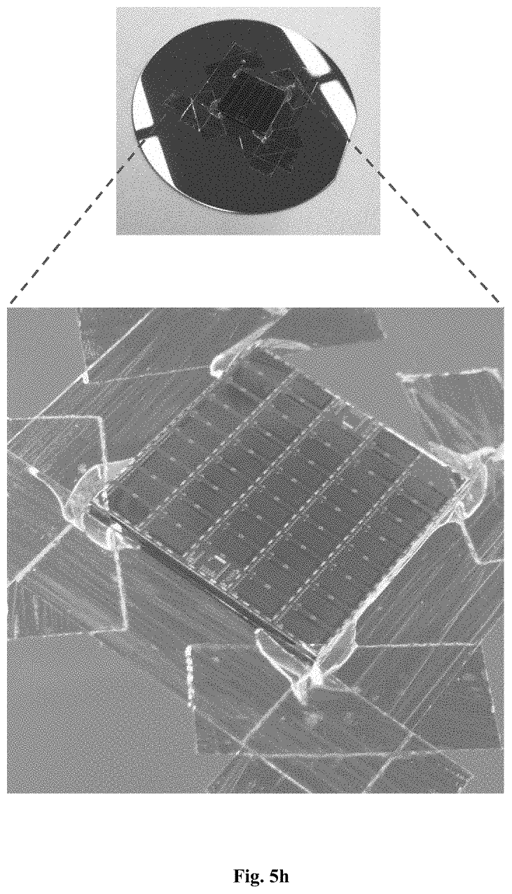

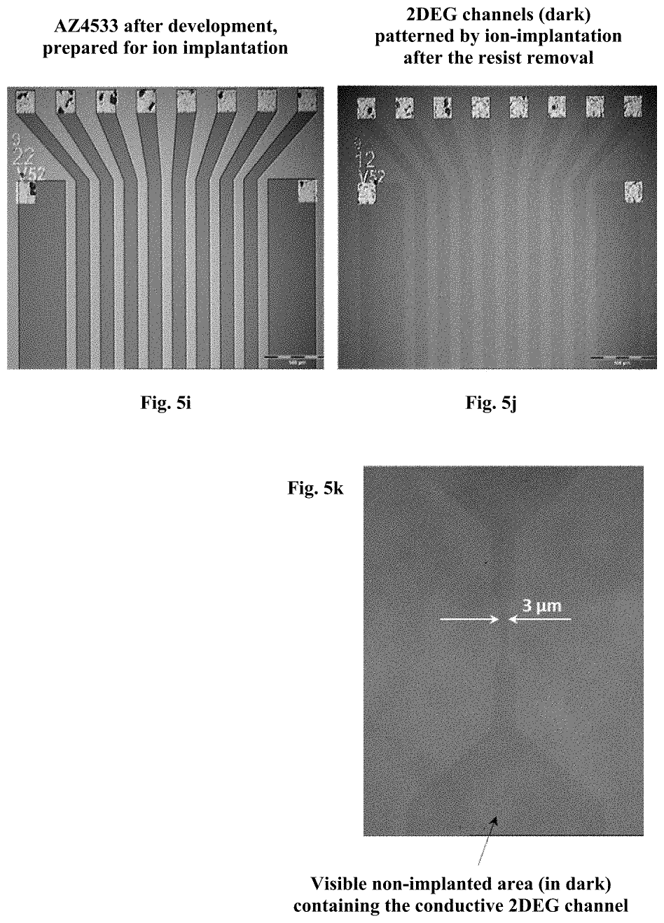

Reference is now made to FIGS. 5a-5c showing the sensor, which is obtained in Step 4 of the 2DEG-channel pattering. The lithography of the sensor was performed with AZ 4533, which is a positive thick resist having optimised adhesion for common wet etching. The lithographic resist film thickness obtained at 7000-rpm spin speed and at 100° C. for 1 min was 3 μm. Thus, as seen in the lithographic image of FIG. 5c, the formed 2DEG channel (13) is approximately 2-3 μm wide. The overall exposure time was 9 sec, followed by 5-min development in MIF726 developer.

FIG. 5d-5e show the mask and corresponding lithographic image, respectively, of the sensor layout of the present invention. FIG. 5f demonstrates the high alignment precision of ±2-μm on 25×25 mm² samples in the lithography of the sensor layout of the present invention. FIG. 5g shows the lithographic images of the multichannel samples. FIG. 5h shows the fixed sensor chip sample on the Si—GaN/AlGaN wafer, which contains approximately 30-32 sensors with 4-8 channels on each sample and prepared for ion implantation. FIG. 5i shows the obtained lithographic image of the present sensor layout with the AZ4533 resist after development, prepared for ion implantation. FIG. 5j shows the 2DEG channels (dark) patterned by ion-implantation after the resist removal. The argon-ion implantation was conducted with 20 keV and 30 keV energies and with an exemplary dose of 2.5e¹³/cm² and a 7° tilt angle. AZ4533 was removed with oxygen plasma at 220 W for 10 min. FIG. 5k shows the visible non-implanted area containing the conductive 2DEG channel.

The atomic layer etching (ALE) performed in Step 8 of the manufacturing process is the most important stage in the process. As mentioned above, it allows the controlled recess of a top layer, removing a single atomic layer-by-layer, where the etch thickness is in the order of magnitude of a single atomic monolayer. As explained above, such ultra-low damage to the top layer of the heterogeneous structure, when the actual surface roughness is controlled by a single atomic monolayer, allows to achieve the sub-nanometre roughness (about 0.2 nm and less) of the top layer when its thickness is only few nanometres (5-9 nm). There are no known ways in the semiconductor technology which would allow to achieve such low roughness at this particular thickness of the semiconductor layer. Therefore, the manufacturing method developed by the present inventors is unique and made it possible to unexpectedly arrive to the pseudo-conducting structures of the present invention.

The ALE process sequence consists of repeated cycling of process conditions. The total amount of material removed is determined by the number of repeated cycles. Each cycle is typically comprised of four steps: adsorption, first purge, desorption and second purge. During the adsorption step of the cycle, reactive species are generated in the reactor (for example, upon plasma excitation), adsorbed by, and react with material on the wafer. Due to the self-limiting process, and with the proper choice of reactants and process conditions, reaction takes place with only a thin layer of material, and the reaction by-products are formed. This step is followed by purging of the reactor to remove all traces of the reactant. Then the by-product desorption takes place due to bombardment of the wafer surface by noble gas ions with a tightly controlled energy. Again, by-products are purged from the reactor, and the wafer is ready for the last two (optional) steps of the manufacturing process.

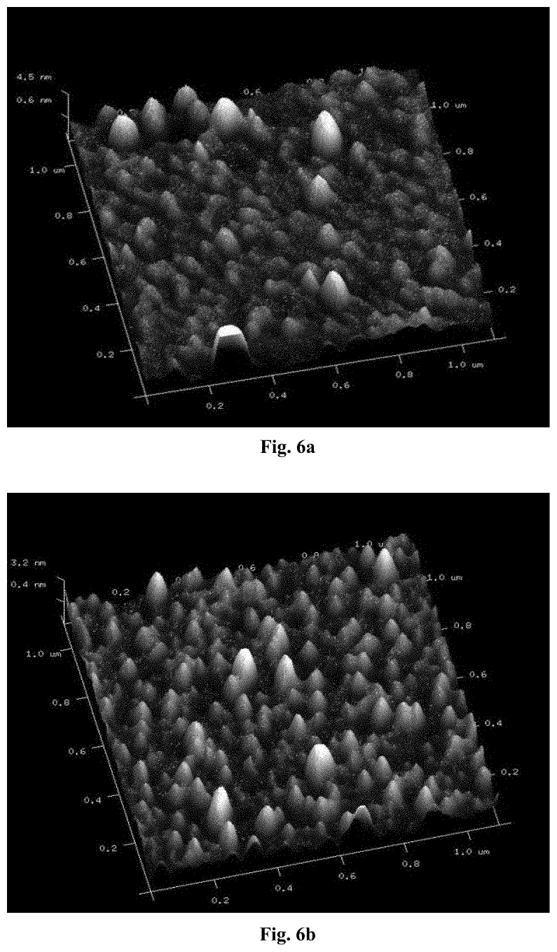

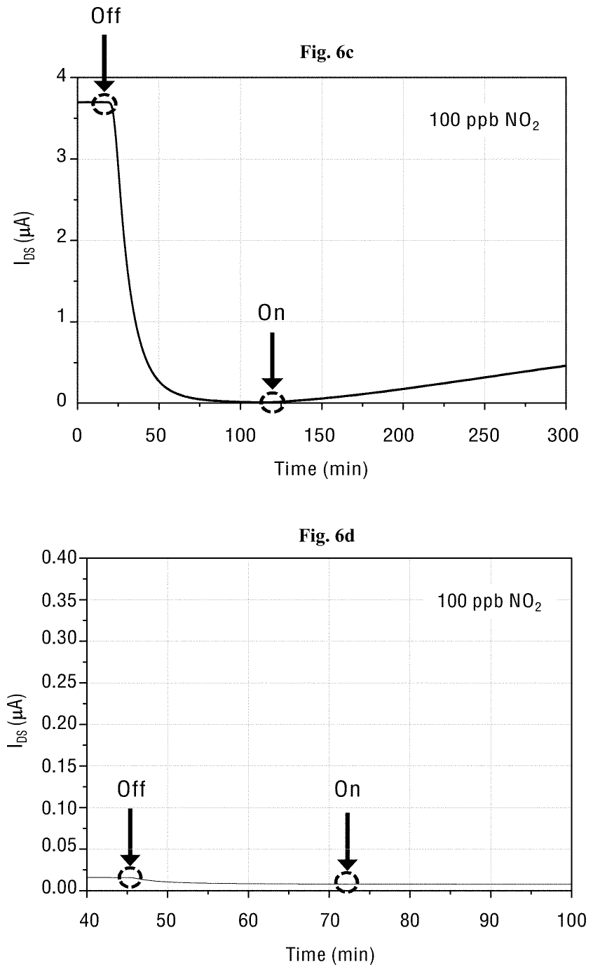

Reference is now made to FIG. 6a showing the AFM image of the top recessed layer surface of the PC-HEMT produced by the manufacturing process of the present invention. The measured RMS value of the surface roughness is 0.674 nm in this case. FIG. 6b shows the AFM surface image of the top recessed layer of the HEMT made by a conventional manufacturing process. In this conventional process, the HEMT initially had a top ultrathin-grown AlGaN layer of the 6-7 nm thickness. This layer was recessed with inductively-coupled plasma (ICP) for 60 sec using a conventional reactive-ion etching (ME) technique. The measured RMS value of the surface roughness is 1.211 nm in this case. FIG. 6c show the time-dependent plot of the drain-source electric current I_{DS} of the nitrogen oxide sensor measuring 100 ppb of the NO₂ gas in 80%-humid air, where the sensor incorporates the PC-HEMT made by the manufacturing process of the present invention. FIG. 6d show the time-dependent plot of the I_{DS} of the nitrogen oxide sensor measuring 100 ppb of the NO₂ gas in 80%-humid air, where the sensor incorporates and based on the HEMT made by the conventional manufacturing process. It is clear from these comparative examples that the manufacturing process of the present invention based on the ultra-low damaging RIE with a narrow plasma-ion energy distribution leads to much lower roughness of the semiconductor surface, which in turn leads to incredibly high sensitivity of the sensor.

In a further aspect, the hetero-junction structure may be a three-layer structure consisting of two GaN layers and one AlGaN layer squeezed between said buffer layers like in a sandwich, wherein the top layer is a buffer layer. This may lead to formation of the two-dimensional hole gas (2DHG) in the top GaN layer above the AlGaN layer which results in reversing polarity of the transistor compared to the two-layer structure discussed above.

In general, polarity of III-V nitride semiconductor materials strongly affects performance of the transistors based on these semiconductors. The quality of the wurtzite GaN materials can be varied by their polarity, because both the incorporation of impurities and the formation of defects are related to the growth mechanism, which in turn depends on surface polarity. The occurrence of the 2DEG/2DHG and the optical properties of the hetero junction structures of nitride-based materials are influenced by the internal field effects caused by spontaneous and piezo-electric polarizations. Devices in all of the III-V nitride materials are fabricated on polar {0001} surfaces. Consequently, their characteristics depend on whether the GaN layers exhibit Ga-face positive polarity or N-face negative polarity. In other words, as a result of the wurtzite GaN materials polarity, any GaN layer has two surfaces with different polarities, a Ga-polar surface and an N-polar surface. A Ga-polar surface is defined herein as a surface terminating on a layer of Ga atoms, each of which has one unoccupied bond normal to the surface. Each surface Ga atom is bonded to three N atoms in the direction away from the surface. In contrast, an N-polar surface is defined as a surface terminating on a layer of N atoms, each of which has one unoccupied bond normal to the surface. Each surface N atom is also bonded to three Ga atoms in the direction away from the surface. Thus, the N-face polarity structures have the reverse polarity to the Ga-face polarity structures.

As described above for the two-layer heterojunction structure, the barrier layer is always placed on top of the buffer layer. The layer which is therefore recessed in the two-layer heterojunction structure is the barrier layer, specifically the AlGaN layer. As a result, since the 2DEG is used as the conducting channel and this conducting channel is located slightly below the barrier layer (in a thicker region of the GaN buffer layer), the hetero-junction structure is grown along the {0001}-direction or, in other words, with the Ga-face polarity. However, as explained above, the physical mechanism that leads to the formation of the 2DEG is a polarisation discontinuity at the AlGaN/GaN interface, reflected by the formation of the polarisation-induced fixed interface charges that attract free carriers to form a two-dimensional carrier gas. It is a positive polarisation charge at the AlGaN/GaN interface that attracts electrons to form 2DEG in the GaN layer slightly below this interface.

As noted above, polarity of the interface charges depends on the crystal lattice orientation of the hetero-junction structure, i.e. Ga-face versus N-face polarity, and the position of the respective AlGaN/GaN interface in the hetero-junction structure (above or below the interface). Therefore, different types of the accumulated carriers can be present in the hetero-junction structure of the embodiments.

In case of the three-layer hetero-junction structure, there are four possible configurations:

### Ga-Face Polarity

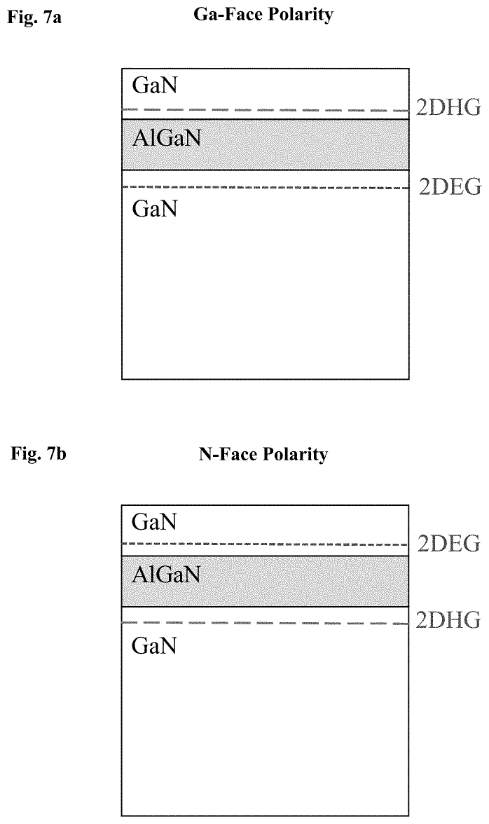

1) The Ga-face polarity is characterised by the 2DEG formation in the GaN layer below the AlGaN barrier layer. This is actually the same two-layer configuration as described above, but with addition of the top GaN layer. In this configuration, the AlGaN barrier layer and two GaN layers must be nominally undoped or n-type doped.
2) In another Ga-face configuration shown in FIG. 7a, in order to form the conducting channel comprising a two-dimensional hole gas (2DHG) in the top GaN layer above the AlGaN barrier layer in the configuration, the AlGaN barrier layer should be p-type doped (for example, with Mg or Be as an acceptor) and the GaN buffer layer should be also p-type doped with Mg, Be or intrinsic.

### N-Face Polarity

3) The N-face polarity is characterised by the 2DEG formation in the top GaN layer above the AlGaN barrier layer, as shown in FIG. 7b. In this case, the AlGaN barrier layer and two GaN buffer layers must be nominally undoped or n-type doped.
4) The last configuration assumes that the 2DHG conducting channel is formed in the buffer GaN layer below the AlGaN barrier layer. The top GaN layer may be present (three-layer structure) or not (two-layer structure) in this case. The AlGaN barrier layer must be p-type doped (for example, with Mg or Be as an acceptor) and the bottom GaN layer should be also p-type doped with Mg, Be or intrinsic.

Thus, there are four hetero-junction three-layer structures implemented in the transistor of the embodiments, based on the above configurations:
A. Ga-Face GaN/AlGaN/GaN heterostructure with the 2DEG formed in the GaN buffer layer below the AlGaN barrier layer. In this case, the top GaN layer may be omitted to obtain the two-layer structure. For the three-layer structure, the top GaN layer must be recessed to 1-9 nm thickness in the open gate area or grown with this low thickness, with the roughness below 0.2 nm, and the thickness of the AlGaN barrier can be adjusted properly during growth
B. Ga-Face GaN/AlGaN/GaN heterostructure with the 2DHG conducting channel formed in the top GaN layer above the AlGaN barrier layer. The top GaN layer must be recessed to 5-9 nm thickness in the open gate area with the roughness below 0.2 nm, and the thickness of the AlGaN barrier layer can be adjusted properly. P-type doping concentrations of the GaN layer and AlGaN barrier have to be adjusted; the 2DHG has to be contacted (in the ideal case by ohmic contacts).
C. N-Face GaN/AlGaN/GaN heterostructure with the 2DEG in the top GaN layer above the AlGaN barrier layer. The top GaN layer must be recessed to 5-9 nm thickness in the open gate area with the roughness below 0.2 nm. Thickness of the AlGaN barrier can be adjusted during growth. N-type doping levels of the GaN buffer layer and the AlGaN barrier layer must be adjusted; the 2DEG has to be contacted (in the ideal case by ohmic contacts).
D. N-Face GaN/AlGaN/GaN heterostructure with the 2DHG in the GaN buffer layer below the AlGaN barrier layer. In this case, the top GaN layer may be omitted to obtain the two-layer structure. In both, the two-layer and three-layer configurations, the top GaN layer must be recessed to 1-9 nm thickness in the open gate area with the roughness below 0.2 nm, and the thickness of the AlGaN barrier can be adjusted properly.

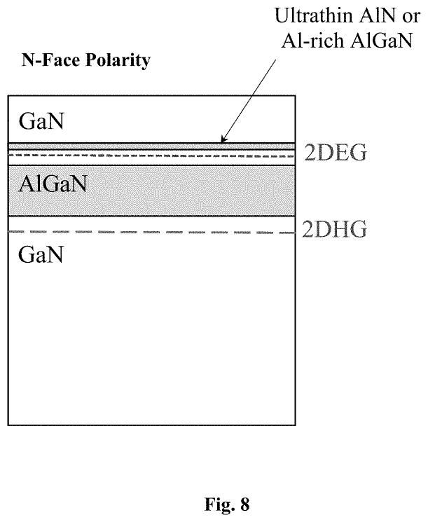

In all the above structures, the deposition of a dielectric layer on top might be beneficial or even necessary to obtain a better confinement (as in case of the N-face structures). As shown in FIG. 8, for the above “C” structure, it may be even more beneficial to include an ultrathin (about 1 nm) AlN or AlGaN barrier layer with high Al-content on top of the 2DEG channel to improve the confinement.

The preferable structures of the embodiments are structures “B” and “C”. In the structure “B”, the 2DHG conducting channel formed in the top GaN layer, which has a higher chemical stability (particularly towards surface oxidation) than the AlGaN layer. Concerning the structure “C”, the 2DEG conducting channel might be closer to the surface. Therefore, the electron mobility might be lower than in the 2DEG structure with the Ga-face polarity. In general, the polarity of the heterostructure can be adjusted by the choice of the substrate (e.g. C-face SiC) or by the growth conditions.

Based on the above, one of the aspects of the present application is an open-gate pseudo-conductive high-electron mobility transistor (PC-HEMT) for performing a sorbent assay, in particular In-Cell ELISA, and for measuring cell dynamics, comprising either:
1) one AlGaN barrier layer at the top of the structure recessed in the open gate area to the thickness of 5-9 nm with the surface roughness of 0.2 nm or less, and one GaN buffer layer at the bottom of the structure; said layers having Ga-face polarity, thus forming the two-dimensional electron gas (2DEG) conducting channel in said GaN layer, close to the interface with said AlGaN layer; or
2) one GaN layer at the top of the structure recessed in the open gate area to the thickness of 5-9 nm with the surface roughness of 0.2 nm or less, one GaN buffer layer at the bottom of the structure, and one AlGaN barrier layer in between; said layers having the Ga-face polarity, thus forming the two-dimensional hole gas (2DHG) conducting channel in the top GaN layer, close to the interface with said AlGaN barrier layer; or
3) one GaN layer at the top of the structure recessed in the open gate area to the thickness of 5-9 nm with the surface roughness of 0.2 nm or less, one GaN buffer layer at the bottom of the structure, and one AlGaN barrier layer in between; said layers having the N-face polarity, thus forming the two-dimensional electron gas (2DEG) conducting channel in the top GaN layer, close to the interface with said AlGaN barrier layer; or
4) one AlGaN barrier layer at the top of the structure recessed in the open gate area to the thickness of 5-9 nm with the surface roughness of 0.2 nm or less, and one GaN buffer layer at the bottom of the structure; said layers having N-face polarity, thus forming the two-dimensional hole gas (2DHG) conducting channel in said GaN layer, close to the interface with said AlGaN layer.

The PC-HEMT of the present invention may further comprise a Vivaldi antenna electrode placed on the top layer between said source and drain contact in the open gate area of the transistor in order to enable detection of electrical signals in the frequency range of 30 GHz to 300 THz. Alternatively, the PC-HEMT of the present invention may additionally comprise (instead of the Vivaldi antenna) a metamaterial electrode, such as an Aharonov-Bohm antenna electrode placed on the top layer between said source and drain contact in the open gate area of the transistor in order to enable detection of electrical signals in the frequency range of 30 GHz to 300 THz. These types of the PC-HEMTs (with a Vivaldi antenna electrode or metamaterial electrode) are described in co-pending patent application U.S. Ser. No. 16/793,839 and PCT/IB2020/051836, respectively, incorporated herein by reference.

As mentioned above, an electrical connection of the multilayer heterojunction structure to the 2DEG or 2DHG channel can be realised via capacitive coupling to the electrical metallisations through a Schottky barrier contact. In that case, since the source and drain contacts are non-ohmic (i.e. capacitively-coupled), the DC readout cannot be carried out. Therefore, in order to electrically contact the 2DEG/2DHG channel underneath, about 5-20 nm bellow the electrical metallisations, the AC-frequency regime must be used. In other words, the AC readout or impedance measurements of the electric current flowing through the 2DEG/2DHG-channel should be performed in this particular case. The capacitive coupling of the non-ohmic metal contacts with the 2DEG/2DHG channel becomes possible only if sufficiently high AC frequency, higher than 30 kHz, is applied to the metallisations. To sum up, the electrical metallisations, which are capacitively coupled to the 2DEG/2DHG channel utilise the known phenomenon of energy transfer by displacement currents. These displacement currents are induced by existing electrical fields between the electrical metallisations and the 2DEG/2DHG conducting channel operated in the AC frequency mode through the Schottky contact as explained above.

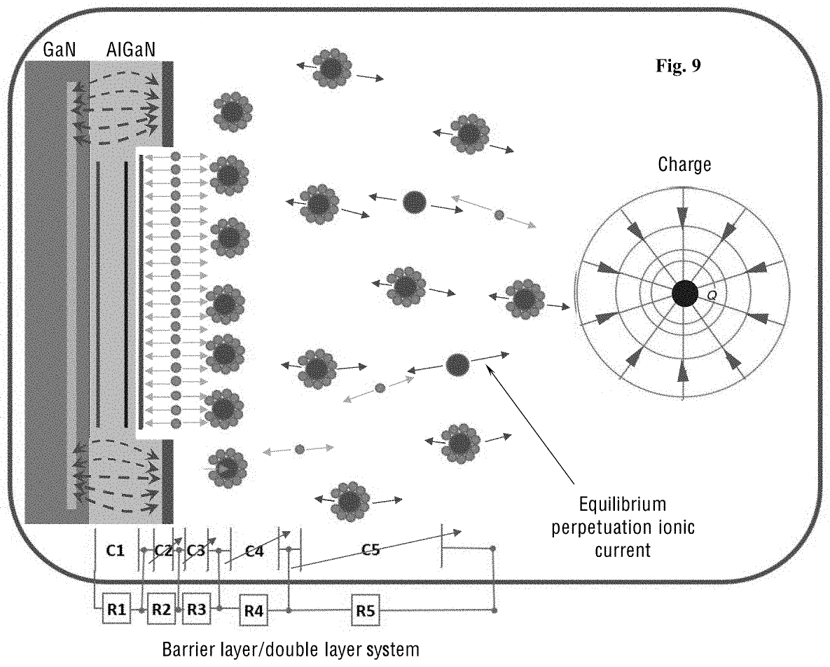

Reference is now made to FIG. 9 illustrating the barrier layer/liquid or gas interface with the double layer formation, simplified equivalent interface circuitry and ion electrodynamics during exposure of the sensor to a charge (positive or negative). When immersed into a gas or liquid environment, any surface potential causes natural formation of an electrochemical double layer at the contact interface to maintain charge equilibrium between the solid state and ionic conductive liquid or gas.

In FIG. 9, this double layer is shown together with the simplified equivalent circuitry at the interface. The double layer is created with a 1- to 3-nm-thick sharp separation between the negative and positive ion space charge zones C2-R2 and C3-R3, which cause a secondary space charge equilibrium zone C4-R4 (10 nm to 1 μm) and charge gradient zone C5-R5 disappearing in the bulk liquid or gas. When there is no more potential shift from the solid and from the liquid or gas, then the charge equilibrium is maintained with C1/R1-05/R5 elements possessing a quasi-constant value.

Ion flow is illustrated in FIG. 9 with the vector arrows during an electrodynamic rearrangement when an external charge is introduced into an equilibrated electrolyte. The arrows in one direction show the electrodynamic rearrangement with an external positive charge, and the arrows in an opposite direction illustrates the electro-dynamic rearrangement but with an external negative charge. When the ions react to an external electric field applied in the liquid, the equivalent circuitry mirroring the space charges changes accordingly. Since the PC-HEMT of the present application is extremely sensitive to any smallest surface charge changes (C1/R1) due to its pseudo-conductivity, as explained above, rearrangement of the gradient ions in the shown space charge zones from C5/R5 to C2/R2 is capable of modulating the 2DEG conductivity. Dynamics and magnitude of the newly formed equilibrium at each time moment is directly proportional to the liquid electrolyte conductivity, ions mobility and external charge value, therefore defining the resulting electrolyte charge. In general, any electrolyte strongly enhances the sensor charge response due to the excellent direct charge transfer towards the barrier layer/electrolyte interface. The ions of the liquid or gas interact directly with the super sensitive surface trap states of the ultrathin barrier layer.

The above phenomenon occurring at the PC-HEMT surface, discovered by the present inventors, is defined as an “intra-fluid ionic” interaction or formation of an “ionic cloud”. Thus, if the PC-HEMT connected to a circuit is immersed into an ion conductive fluid (being liquid or gas), then ions of the fluid start electro-dynamically react to any external charge by their movement and form the ionic cloud. Being in direct contact or close proximity to the barrier layer surface, the charge sensitivity is thus tremendously enhanced by this ionic cloud. The fluid actually acts in this case as an antenna matching the 2DEG transducer perfectly. Electric charges generated in any environment, as well as their super position dipoles forming the ionic cloud, are projected to this fluid antenna, in which the transistor is immersed. Sensing of the electric charges with the PC-HEMT of the present invention is therefore possible in a contactless manner, when the molecules are at some distance from the surface of the transistor. This clearly allows to overcome a usual “sensing noise” of any traditional biosensor having reporter molecules attached to the surface of the sensor, as will be discussed below.

To sum-up, in a direct current (DC) mode, the top layer-to-fluid-interface of the PC-HEMT is in charge equilibrium, where the 2DEG is directly incorporated as a balancing polarisation element. Once an external electric charge (originated from dipole molecules forming a layer or an ionic cloud, or two neutral molecules creating a dipole pair under London forces and also forming an ionic cloud) is introduced into the electrolyte fluid environment, the net charge equilibrium is shifted resulting in a change in the electron density and mobility. In case of the pseudo-conducting 2DEG channel, it becomes easily modulated and the strongest amplification phenomenon is observed. Sensitivity of the PC-HEMT in this case is so ultra-high that it allows detecting neutral molecules diffusing to the surface and coupling to the PC-HEMT top layer surface via a getter effect changing the surface trap states. The getter effect actually allows the sensor based on the PC-HEMT of the present invention to collect free gases by adsorption, absorption or occlusion.

In a radiofrequency (RF) mode, when the electric current in the 2DEG/2DHG channel is alternating (AC), the near-field and displacement current coupling effects at electrochemical double layers take place. In that case, the super-Debye interactions allow detection of any ion types selectively at MHz frequency range and ion solvation shells and resonance frequencies of intra-fluid ion-ion interaction at GHz range.

As mentioned above, in most cases (in most biosensors), molecular receptors bound to the transistor surface are spatially separated from this surface by molecular cross-linkers or proteins of approximately 5-15 nm length. Therefore, the aforementioned charges are screened from the sensing surface by dissolved counter ions. As a result of the screening, the electro-static potential that arises from charges on the analyte molecule exponentially decreases to zero with increasing the distance from the sensing surface. This screening distance is defined as a “Debye length”, and it must be carefully selected when designing the receptor layer of any ISFET in order to ensure the optimal sensing. For example, when detecting molecules in blood serum, the typical Debye screening length is 0.78 nm at temperature 36° C. with an electrical permittivity of 74.5 for water. The screening length means that an electrical field originating from a point charge is dropped to its 1/e value (29%) in this length. Because of this limitation, charges from larger biomolecules (5-15 nm) cannot be detected in a serum sample. To overcome this problem, the charges should be attracted closer to the sensor surface by using very short length receptors or by operating the sensor in completely desalted buffers for electronic molecular detection.

The present inventors surprisingly found that it is actually possible to sense beyond the Debye screening length, without any modification of the receptors and even without any functionalisation of the top layer surface of the PC-HEMT with (bio)molecular receptors, by operating the PC-HEMT of the present invention at high frequencies and using a combined transducer principle. The present inventors have recently discovered that the precise identification of biomolecules and cells can be obtained by combination of a precise monitoring and control of the main parameters, temperature, pH and ionic strength with an array of electronically identical PC-HEMTs of the present invention.

Since the main limitation in the DC readout mode is the Debye screening of charges, the DC alone is not really suitable for sensing of biomolecules and cells and mainly depends on the charge carried by the target biomolecules. The present inventors proposed to overcome these limitations by adding the AC readout with a frequency sweep up to 1 MHz or higher. Opposite to the DC readout, the charges of the target molecules have a negligible influence on the sensing in the AC mode.

In addition, the AC readout can also detect the presence of the bound molecules or cells. The AC sensing mode has the same basis as the impedance spectroscopy. It shows the change of the sensor's surface capacitance and resistance which contains information about the binding of the target molecule, as well as the ‘number’ (concentration) of the bound molecules or cells.

Thus, the AC electronic readout combined with the DC readout is useful for enzymatic, electrochemical and affinity sensing when both charged and uncharged molecules are involved. When operated at higher frequencies (more than 1 MHz), the problematic Debye screening can be overcome, and also larger molecules can be sensed.

In a further aspect of the present invention, the combined transducer principle defined herein as a “multiparametric readout” includes: DC electronic readout of the sensor, AC electronic readout of the sensor and temperature sensing. The PC-HEMT-based sensor of the present invention therefore further comprises a reference or counter electrode and characterised with respect to its electronic properties and the measurement configuration for molecular sensing applications. The main features of the sensors of the present invention are determined by the transfer characteristics and the output characteristics at room temperature. The transfer characteristics shows the drain current of the transistors as a function of their source voltage at constant drain-source voltages.

In general, the term “transfer function” (TF) is a mathematical representation to describe inputs and outputs of black box models. In order to describe the frequency response of the sensor, a counter electrode and the first amplifier stage are considered as a black box element with a certain frequency response. Since the analogue transistor amplification is exploited in the present invention, the instant model is described with a term “transistor transfer function” (TTF). The TTF is defined as a mathematical ratio between the input (Vₛₜᵢₘ) and the output signal (Vₒᵤₜ) of an electrical, frequency-dependent system. Its frequency response H(jω) is defined as follows:

$$H ( j ω ) = \frac{{V}_{out} ( j ω )}{{V}_{stim} ( j ω )} ,$$

wherein ω is the angular frequency and j is the imaginary unit.

Thus, the TTF can be used to investigate impedance (defined as the ratio between voltage and applied current) or capacitance (defined as the capability of a capacitor to store charges) changes, caused by binding of molecules or cells onto the surface of the transistor.

The DC electronic readout is based on the transfer characteristics and is carried out in a liquid medium. The sensor in a DC readout mode is biased by a certain drain-source voltage while a voltage sweep is done through a reference electrode, and it senses the charges at the sensor surface. The resulting transfer characteristics reflect the characteristic behaviour of the sensor, as well as its surface condition, and are used to detect target molecules or cells on the sensor surface. In addition to the DC mode, the AC electronic readout is used to enable the multiparametric readout. When operated at higher frequencies (more than 1 MHz), the Debye screening can then be overcome, and also larger molecules and cells can be sensed.

To sum up, at any solid state/electrolyte interface, the capacitive and resistive elements of the sensor form an electrochemical surface potential originated from an interaction between the surface trap states and a double layer capacity, while the interaction between the 2DEG and the surface trap states originates from tunneling and electrostatics. It has now been surprisingly found that operation of the PC-HEMT sensor as an open gate field-effect transistor is not required in order to modulate the surface electrochemical potential within the barrier layer/electrolyte system.

In a specific embodiment, the microelectronic sensor of the present invention for performing sorbent, immunosorbent, sorbent or cell assays and for measuring cell dynamics comprises the following components:
(a) the PC-HEMT of the present invention, or an array thereof, wherein each of said transistors is connected to its dedicated electrical contact line (thereby constituting a sensing channel);
(b) a voltage source connected to said electrical contact lines via an electric circuit for supplying electric current to said transistors;
(c) an integrated or CMOS current amplifier connected to said voltage source for amplification of an electric current obtained from said transistor/s;
(d) an analogue-to-digital converter (ADC) with in-built digital input/output card connected to said current amplifier for converting the received analogue signal to a digital signal and outputting said digital signal to a microcontroller unit;
(e) the microcontroller unit (MCU) for processing and converting the received digital signal into data readable in a user interface or external memory; and
(f) a wireless connection module for wireless connection of said microelectronic sensor to said user interface or external memory.

The ADC card may be any suitable analogue-to-digital converter data logger card that can be purchased, for example, from National Instruments® or LabJack®. Optionally, the current amplifier can be operated directly with current flowing via the conducting 2DEG channel into the amplifier with small input resistance of 1MΩ at gain higher than 10⁴ and only 1Ω at gains lower than 200. This setup may directly amplify the electric current modulation in the 2DEG channel originated from external body charges.

In a specific embodiment, the wireless connection module may be a short-range Bluetooth® or NFC providing wireless communication between the sensor and the readout module for up to 20 m. If the connection module is Wi-Fi, the connection can be established with a network for up to 200 nm, while GSM allows the worldwide communication to a cloud. The external memory can be a mobile device (such as a smartphone), desktop computer, server, remote storage, internet storage or cloud.

Alternatively, the PC-HEMT of the invention may be based on a piezoelectric electro-optical crystal (EOC) transducer. The PC-HEMT based on the EOC piezoelectric substrate exhibits the highest coupling between electrical and mechanical energy compared to all other varieties of substrates. Additionally, such a substrate also has the advantages of having a high velocity-shift coefficient and a very high electromechanical coupling coefficient, K2, which yields a greater mass sensitivity in comparison with the same regular SAW device on any other piezoelectric substrates. The EOC may be any suitable electro-optical crystalline material such as LiNbO₃, which is brought into a contact with a medium to be sensed. The EOC is then illuminated with a polarised light.

In case of the LiNbO₃ crystalline material, the wavelength of the polarised light is about 400-600 nm. Modulated light from the light source illuminates the substrate with the EOC, and then falls on the 2DEG structure. The 2DEG structure is ultrasensitive to an incident light creating the p-n-pairs in the top recessed layer and as a result, strongly affecting the 2DEG conductivity. In general, irradiation of the 2DEG structure with light switches the 2DEG channel from normally-off to a pseudo-conducting or normally-on state. Therefore, being in a close proximity to the ionic cloud, the EOC is capable of changing its light absorbance strongly affecting electrical current in the 2DEG channel, thereby resolving any smallest light intensity changes coming from the EOC transducer. Depending on the excitation light wavelength, the position of the sensor relative to the incident light beam can be changed. For instance, in case of IR light (700-1500 nm), the sensor should be placed perpendicularly to the light beam for achieving the highest sensitivity. The parasitic charging of the EOC is compensated via the electrodes attached to the crystal. Additionally, a variety of light filters in front of the sensor can be utilised. Thus, the use of the EOC configuration of the PC-HEMT of the invention makes it possible to drastically increase sensitivity of the sensor to an electrical charge.

In a further specific embodiment, the microelectronic sensor of the present invention for performing sorbent, immunosorbent, sorbent or cell assays and for measuring cell dynamics comprises the following components:
(i) the PC-HEMT of the present invention, or an array thereof, wherein each of said transistors is connected to its dedicated electrical contact line (thereby constituting a sensing channel);
(ii) a modulated light source, such as a surface-mounted-device light-emitting diode (SMD LED) or UV-VIS-IR laser diode, for irradiating the top layer surface of said transistors;
(iii) a voltage source connected to said electrical contact lines via an electric circuit for supplying electric current to said transistors;
(iv) a lock-in amplifier connected to said voltage source for amplification of a signal with a known carrier wave obtained from said transistors and increasing the signal-to-noise ratio;
(v) an analogue-to-digital converter (ADC) with in-built digital input/output card connected to said lock-in amplifier for outputting the converted signal to a user interface;
(vi) a feedback control microcontroller unit (MCU) for energy level adjustment and de-trapping via an external or integrated gate electrode; and
(vii) a wireless connection module for wireless connection of the sensor to a readout module; wherein said readout module comprises another wireless connection module connecting the sensor to said user interface via a digital-to-analogue converter (DAC).

In some embodiments, the sensors of the present application can be used for portable long-time-operation solution within remote cloud-based service. The portable sensor of an embodiment should have a very small power consumption saving the battery life for a prolong usage. In this case, the non-ohmic high-resistive contacts capacitively connecting the sensor to an electric circuit are preferable. The non-ohmic contacts actually limit an electric current flowing through the 2DEG/2DHG channel by having an electrical resistance 3-4 times higher than the resistance of the 2DEG/2DHG-channel, thereby reducing electrical power consumption without sacrificing sensitivity and functionality of the sensor. Thus, the use of non-ohmic contacts in some embodiments of the sensor of the present application is a hardware solution allowing minimising the power consumption of the device. In another embodiment, the power consumption of the device can be minimised using a software algorithm managing the necessary recording time of the sensor and a battery saver mode, which limits the background data and switches the wireless connection only when it is needed.

In a further embodiment, a method for sorbent, immunosorbent or cell sorbent assay of a sample containing a chemical compound or a biological compound to be tested in a gas phase or in a liquid phase, comprises the following steps:
(1) Subjecting the sample to the microelectronic device of the present invention;
(2) Recording electrical signals received from the said microelectronic device in a form of a source-drain electric current of the microelectronic sensor over time (I_{D}s dynamics);
(3) Transmitting the recorded signals from said microelectronic device to an external memory for further processing; and
(4) Converting the transmitted signals to digital signals and processing the digital signals in the external memory, comparing said I_{DS} dynamics with negative control chemical or biomolecular I_{DS} waveforms stored in the external memory, and extracting biochemical or biomolecular information from said waveforms in a form of readable data, thereby detecting and/or identifying a particular biological compound or cell in the sample and measuring their concentration or amount and biochemical or biophysical parameters.

The sensors of the present invention have been proved to be an effective tool for rapid drug screening with accurate results and in point-of-care diagnostics. The present invention also provides methods for identifying targets of a drug in a cell by comparing the effects of the drug on a cell, the effects on a cell of modifications to a target of the drug, and the effects of the drug on a cell which has had the target modified.

Among other applications, the sensors of the present invention are useful for studying the effect of pharmaceuticals on a human heart. Neuronal and cardiac muscle cells (cardiomyocytes) are commonly used for initial medical trials in pharmacological examinations. To obtain results similar to the response of cells in the human body, human-induced pluripotent stems (iPS) are most often used. To get FDA approval, the new drug should not have a harmful effect on the heart. In addition, the benefits achieved must be greater than the damage done.

The flow of ions in or out of the cell as well as the action potential can be measured by performing electrophysiological techniques as patch-clamping. Calcium flow is an important factor for the physiological function of the human body, as it affects the flow of ions and, therefore, signal transmission. As a result, multiple diseases are caused by impaired calcium metabolism. As already mentioned, drugs can have a negative effect on the human body, for example, by changing the action potential of the cell, which is activated by the flow of ions and which leads to a contraction of the heart. Therefore, it is very important to measure cell contraction, since any pathological contraction indicates impaired cell function. A huge drawback of methods such as patch-clamping is that only the ion flux can be detected, but not the contractility of the cells.

In contrast, the sensors of the present invention can be used to detect the cell dynamics, including contractility of cells. This is done by growing cells directly on the surface of the sensor. Cell movement is measured electronically in real time. In addition, the sensor collects all the information received from the cell signal because the signal is not filtered. This is a great advantage, for example, against optical methods that use specific algorithms to analyse the obtained optical result. Thanks to an electronic readout, immunostaining and expensive measuring equipment are not required to obtain results. In the following experimental examples supporting the present invention, cardiomyocytes are used primarily because they give a strong and clearly defined electrical signal as well as a visible contraction, which can be correlated to that signal.

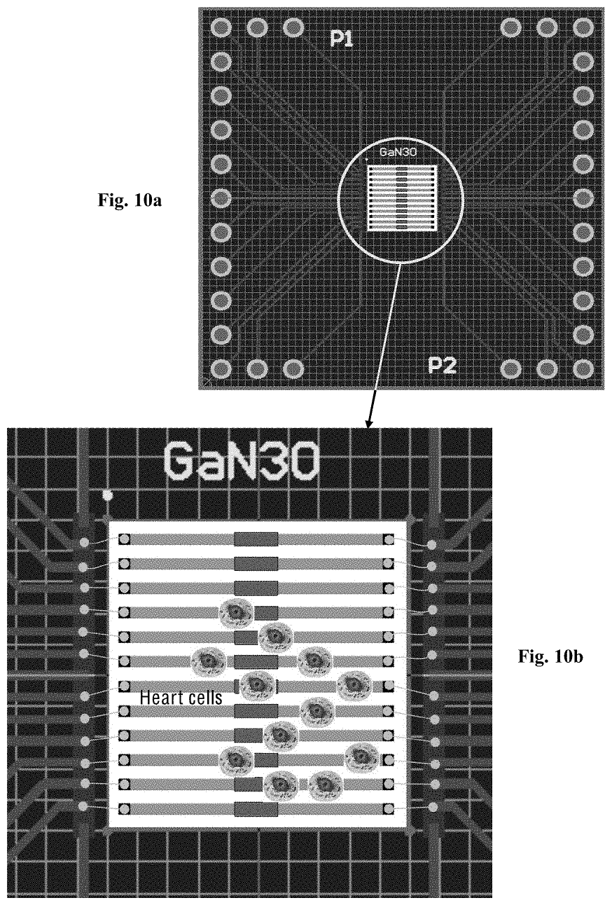

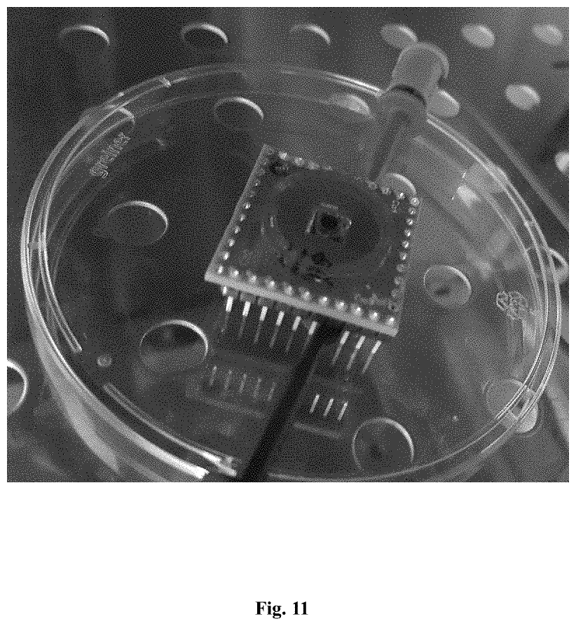

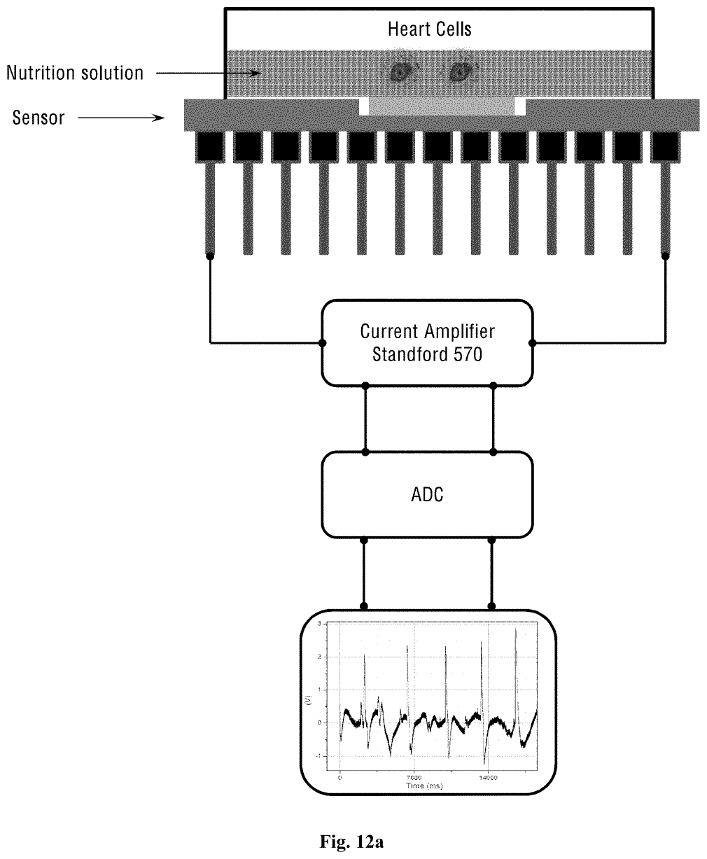

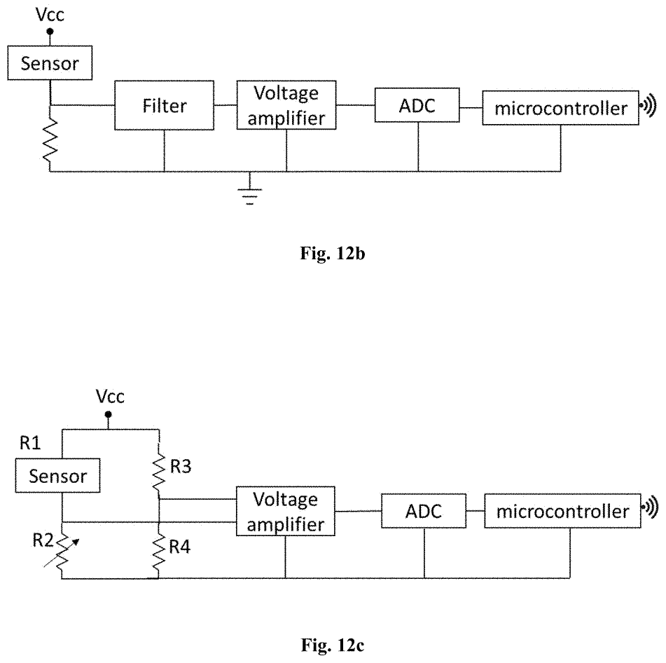

FIGS. 10a-10b show the exemplary sensor layout of the present invention. The exemplary sensor has 2×12 contact pads. The sensor shown in FIGS. 10a and 10b was used in the in-cell sorbent assay in the frame of the cell signalling experiment and for measuring cell dynamics. Cardiac muscle cells (cardiomyocytes) were introduced onto the surface of the sensor. Before applying the cells, the surface of the sensor was thoroughly rinsed with deionised water. The sensor was then sterilised with 70% ethanol under the fume hood until all the solvent has been evaporated. The clean sensor surface was coated with fibronectin as recommended by the cell kit provider. FIG. 11 shows the photograph of this sensor having the layout shown in FIGS. 10a-10b and the 2×12 contact pads (with 12 sensing channels). FIG. 12a schematically shows this sensor and the sensor circuit. The sample containing cardiomyocytes in nutrition solution is dropped on the surface of the sensor.

Cardiomyocytes were digested and isolated from chopped neonatal mouse heart (having age of 1-3 days). They were seeded on the sensor coated with fibronectin. After 3-5 days in culture, cardiomyocytes started beating on the sensor. Depending on the number of cells used (1 k to 10 k per sensor), cells beat in independent clusters (low density) or synchronise completely (high density). The cells stayed active for several weeks. The protocol for cardiomyocytes isolation using enzyme digestion of neonatal heart tissue with the Pierce Primary Cardiomyocytes Isolation Kit is available online at ThermoFischer Scientific®. The active cardiomyocytes were measured under controlled conditions in incubator at 37° C. and 5% CO₂ with the sensor shown in FIGS. 10a-10b, 11 and 12a.

Several electronic configurations of the sensor were used in the present experiments. In the configuration shown in FIG. 12b, the sensor is connected to a resistor in series to create a voltage dividing circuit. The output of the circuit is fed into a high pass, or a band pass filter to filter out the unwanted frequencies and the DC offset. The filtered signal is then fed into a voltage amplifier and digitised by an ADC. The digital signal can be loaded to a microcontroller or microprocessor before transmitting via wireless or wired transmission.

In the configuration shown in FIG. 12b, the sensor of the invention is connected in a resistor bridge network to form a Wheatstone bridge. One or more of the resistors in the network are programmable variable resistors. In one configuration of the present embodiments, the resistors are programmed to have the differential voltage between the two sides of the networks as minimal as possible. The differential signal is then amplified and digitised with an ADC and then fed to the microcontroller before transmitting. The resistors are programmed with the microcontroller forming closed loop feedback. The resistors are programmed as when required to keep the differential voltage minimum such that the output of the amplifier is below the rail to rail voltage.

In another configuration of the present embodiments, the differential voltage is kept zero so that the bridge is always balanced. The resistance changes on the variable resistor to keep the differential voltage zero are proportional to the resistance change of the sensor. In this configuration the voltage amplifier is optional. Microcontroller constantly monitors and adjusts the closed loop feedback to the programmable resistors to keep the bridge balanced.

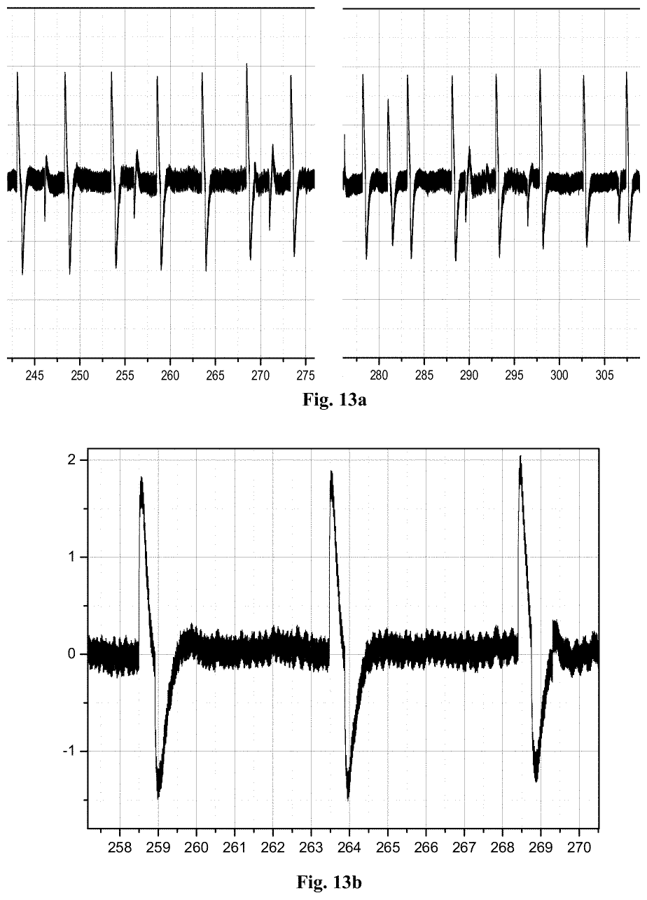

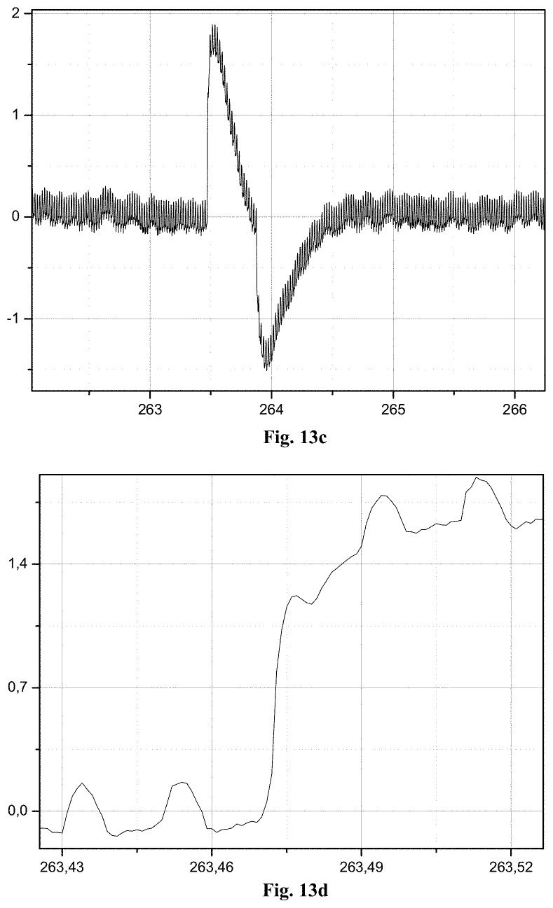

Reference is now made to FIG. 13a showing the I_{DS} dynamics in a continuous readout of the cardiomyocytes on the surface of the sensor with the ultra-high signal to noise ratio. FIG. 13b is the expansion of the I_{DS} dynamics shown in FIG. 13a, whereas FIGS. 13c and 13d are expansions in the narrower ranges showing the fine fingerprint of the beats.

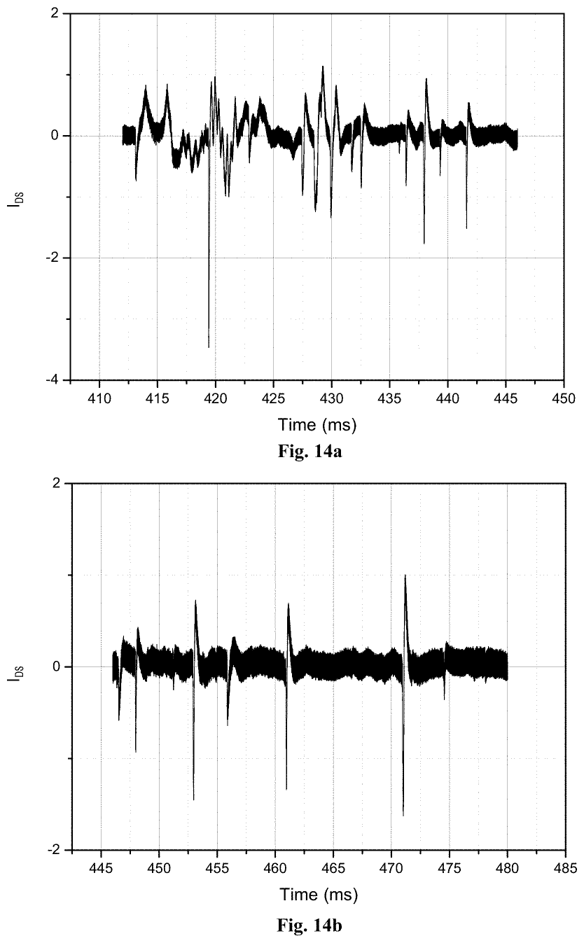

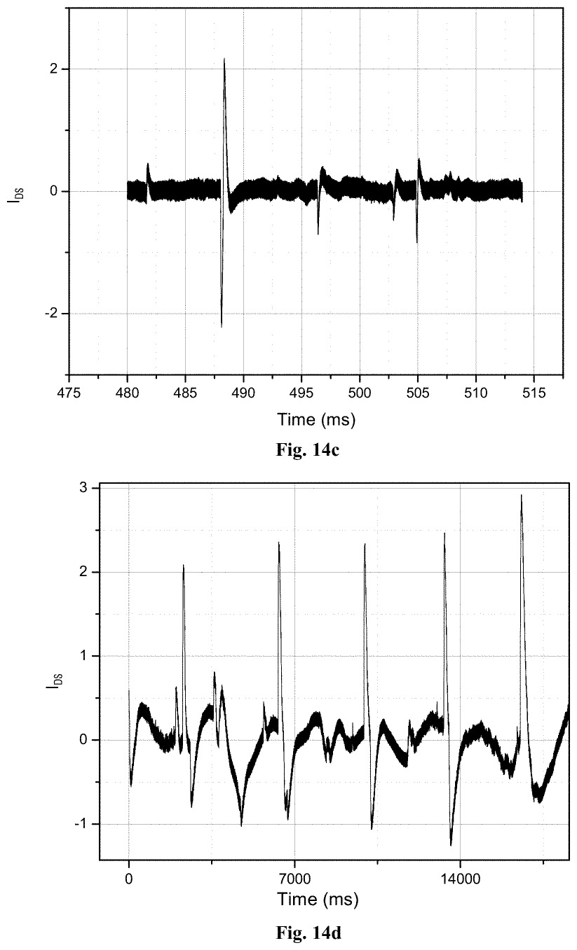

FIGS. 14a-14d show the change in the I_{DS} dynamics of cardiomyocytes upon addition of noradrenaline/norepinephrine and nifedipine to the cardiomyocytes medium together with the nutrient medium change. FIGS. 14a-14c demonstrates the silencing of the cardiomyocytes upon addition of nifedipine (the Ca²⁺-antagonist, its concentration was taken from literature) at different time intervals (410-450 ms, 445-485 ms and 480-515 ms, respectively). The spectra show that the cell signal frequency recorded from the cardiomyocytes is decreased, because the cells are silenced with nifedipine, which leads to this decrease. After silencing the cells, their signals are still observed in the spectra because the cells were not completely knocked out. The cells are actually only silenced, which means that their activity is suppressed and, therefore, the frequency of the signal decreases. FIG. 14d shows further recovering of the cell activity upon addition of noradrenaline, as the signal frequency is increased.

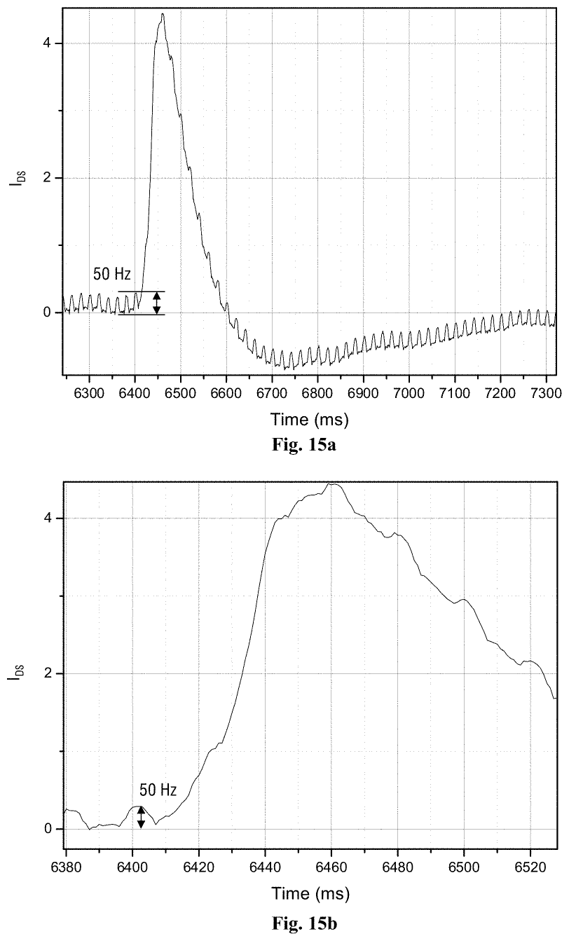

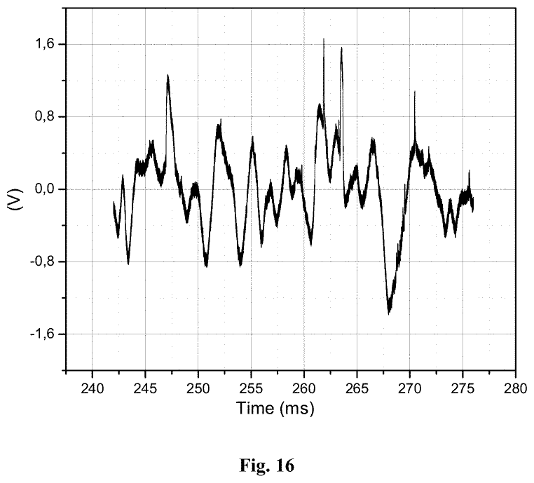

Reference is now made to FIGS. 15a-15b showing the signal-to-noise ratio in the present experiment. As can be clearly seen from these figures, there is practically no intrinsic noise from the sensor (the signal-to-noise ratio is about 1000 or higher). Lastly, FIG. 16 shows the signal recorded by the sensor after cell death and removal from the surface of the sensor. As can be seen, the cell signal disappears after the cell is removed, leaving some noise due to some impurities and waste remaining on the surface after the experiment. Thus, in the above experiment, it was demonstrated that the sensor of the present invention is capable of detecting signals emitted by a single cell or very few cells on its surface and measuring the cell dynamics.

Thus, the present invention provides a method for in-vitro measurement of a cell dynamics, said method comprising:
(1) Subjecting a cell culture to a surface of a microelectronic sensor of the present invention or growing said cell culture directly on the surface of the sensor;
(2) Recording electrical signals received from the cell culture in a form of a source-drain electric current of the microelectronic sensor over time (I_{DS} dynamics) in real time;
(3) Transmitting the recorded electrical signals from said sensor to an external memory for further processing; and
(4) Converting the transmitted signals to digital signals and processing the digital signals in the external memory, comparing said I_{DS} dynamics with negative control I_{DS} waveforms stored in the external memory, and extracting information on cell dynamics from said waveforms in a form of readable data.

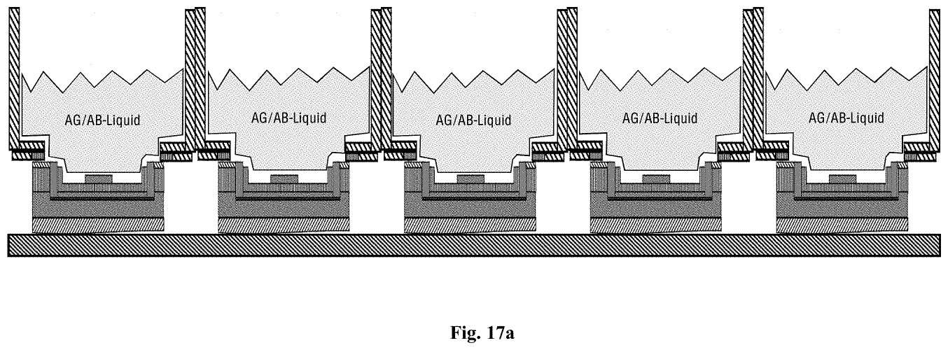

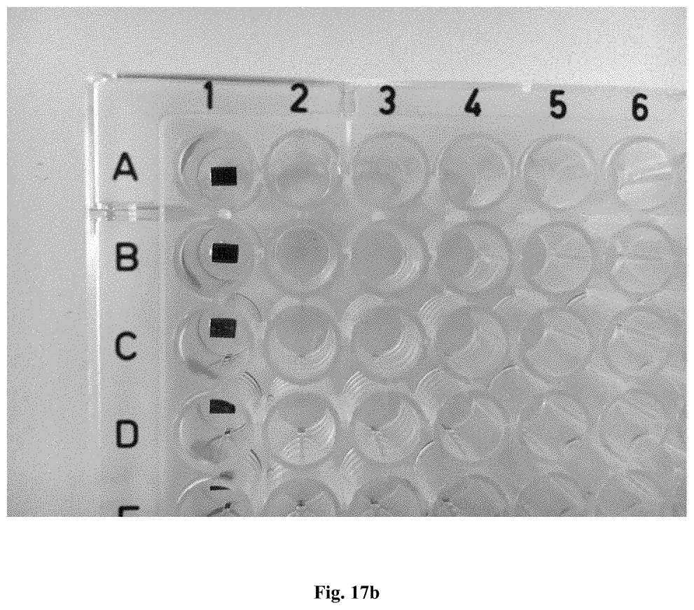

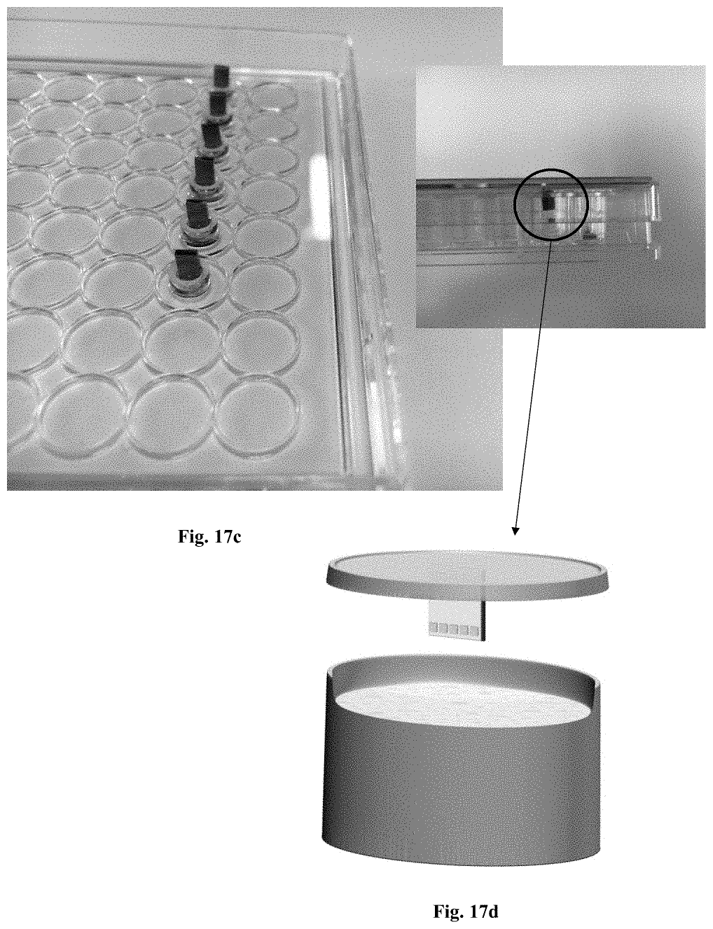

FIG. 17a schematically shows the “Electronic ELISA Plate” of the present invention, incorporating the sensors of the present invention. This is actually a cross-cut through standard 96-wells microwell plate for ELISA where the sensor of the present invention was inserted in each single “well” of the plate (at the bottom of each well), thereby turning each well into a separate electronic test tube for the sorbent assay. FIG. 17b shows this electronic 96-well plate of the present invention, in a bottom-up format. FIGS. 17c and 17d show the image of the electronic 96-well plate of the present invention, in a top-down format, and a schematic enlarged image of a single well from this plate, respectively. The electrical contacts are integrated in the upper microwell plate contact array with the MID (moulded interconnect device) technology. The MID is actually an injection-moulded thermoplastic part with integrated electronic circuit traces. The MID technology combines plastic substrate/housing with electric circuitry into a single part by selective metallisation. Thus, the sensors of the present invention are integrated into the microwell plate via polymer moulding process. The resulting microwell plate is the “Electronic ELISA Microwell plate” of the present invention, which is fully standardised and therefore, compatible, with all common ELISA tools.

In one of the aspects of the present invention, a microelectronic microwell plate for sorbent, immunosorbent or cell sorbent assay comprises:
(1) a plurality of the microelectronic sensors of the present invention, wherein each sensor is integrated at the bottom of its corresponding well of said microwell plate and connected to its dedicated electrical contact in a contact array;
(2) the contact array integrated at the top of said microwell plate;
(3) a row multiplexer connected to said contact array for addressing each and every sensor arranged in rows, selecting one of several analogue or digital input signals and forwarding the selected input into a single line;
(4) a column multiplexer connected to said contact array for addressing each and every sensor arranged in columns, selecting one of several analogue or digital input signals and forwarding the selected input into a single line; and
(5) an integrated circuit for storing and processing said signals.

In some embodiments, a method for sorbent, immunosorbent or sorbent cell assays comprises the following steps:
(a) Subjecting a sample to be tested to the microelectronic microwell plate of the present invention;
(b) Recording electrical signals received from the sample in a form of a source-drain electric current of the microelectronic sensor over time (I_{D}s dynamics);
(c) Transmitting the recorded signals from said microelectronic microwell plate to an external memory for further processing; and
(d) Converting the transmitted signals to digital signals and processing the digital signals in the external memory, comparing said I_{DS} dynamics with negative control chemical or biomolecular I_{DS} waveforms stored in the external memory, and extracting biochemical or biomolecular information from said waveforms in a form of readable data, thereby detecting and/or identifying a particular biological compound or cell in the sample and measuring their concentration/amount and biochemical/biophysical parameters.

### Electronic ELISA Experiments

Electronic ELISA experiments using the sensor of the present invention and supporting the present invention are presented below. In all the electronic ELISA experiments, the assay kits were purchased from EUROIMMUN®. The assay kits contained washing buffer, capture analyte, blocking solution and target analyte. The buffer preparation was not necessary since all necessary buffers were provided by EUROIMMUN®. The composition of these buffers is confidential and cannot be disclosed. All the samples were prepared according to the proprietary protocols provided by EUROIMMUN®. These protocols are also confidential and cannot be disclosed. Each experiment was performed according to the proprietary protocol provided by EUROIMMUN® and cannot be disclosed either.

In general, the following experimental procedure in each experiment was carried out:
1. Rinsing after encapsulation is only done if the sensor surface was extremely dirty. For this purpose, deionised water was used to carefully rinse the chip. If the surface looks good no surface cleaning is done.
2. Positive control: The sensor surface was coated with the capture molecule solution. The concentration was taken from the EUROIMMUN® protocol. The immobilisation was then performed for either 2 hours at room temperature or overnight at 4° C. in the fridge.
3. Negative control: The sensor was incubated with buffer (same buffer, in which the capture analyte was diluted to its final concentration) for either 2 hours at room temperature or overnight at 4° C. in the fridge.
4. The positive and negative sensor were washed very carefully with the assay buffer after the immobilisation had been completed.
5. The positive and negative sensors were blocked with the blocking buffer for 1 hour at room temperature.
6. The positive and negative sensor were carefully washed with the assay buffer afterwards.
7. 40 μL of the assay buffer was introduced onto the sensor surface using a micropipette, and the measurements of both sensors (positive and negative control) were performed in parallel.
8. After 10 min, 40 μL of the target analyte solution (concentration of the target analyte is given in the EUROIMMUN® protocol) was added, and the measurements were continued.
9. After adding the target analyte, the sensor was not washed anymore. The measurements were stopped when the signal had become stable.

In each experiment, two sensors were used either modified or incubated with the positive sample, and two sensors were used either modified or incubated with the negative sample. Volume of measuring cavity in each sensor was 80 μL. Three or four electronic channels were used for each negative or positive sample, and the readout was done simultaneously. Raw I_{DS} data was generated for positive and negative samples. The corrected data was obtained by a baseline correction in each channel, which was done by creating exponential model. The baseline was subtracted from the raw data.

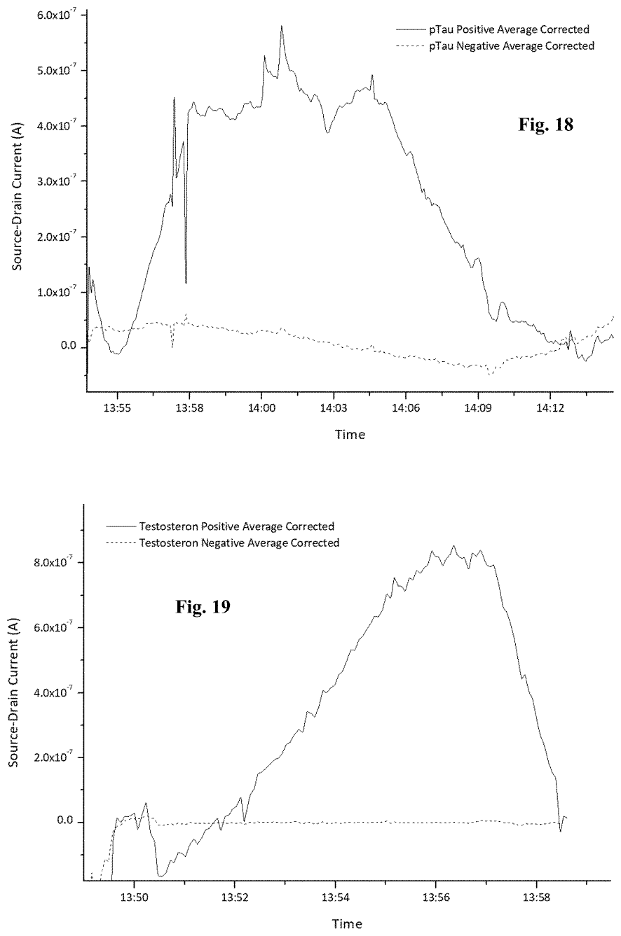

Reference is now made to FIG. 18 showing the corrected plot averaged on four channels for the anti-pTau assay. Tau (τ) protein is a protein that is capable of stabilising microtubules in neurons of the central nervous system. Pathologies and dementias of the central nervous system, such as Alzheimer's disease and Parkinson's disease, are often associated with a tau protein that has become defective and is no longer capable of stabilising microtubules properly. The results of the assay shown in FIG. 18 clearly support the method of the present invention. Normally, it is hard to detect a tau protein due its very small size. However, because of the aforementioned advantages of the electronic ELISA of the present invention, including the enormously high signal-to-noise ratio, extremely high sensitivity and overcoming the Debye length limitation, the positive sample is clearly distinguished from the negative sample.

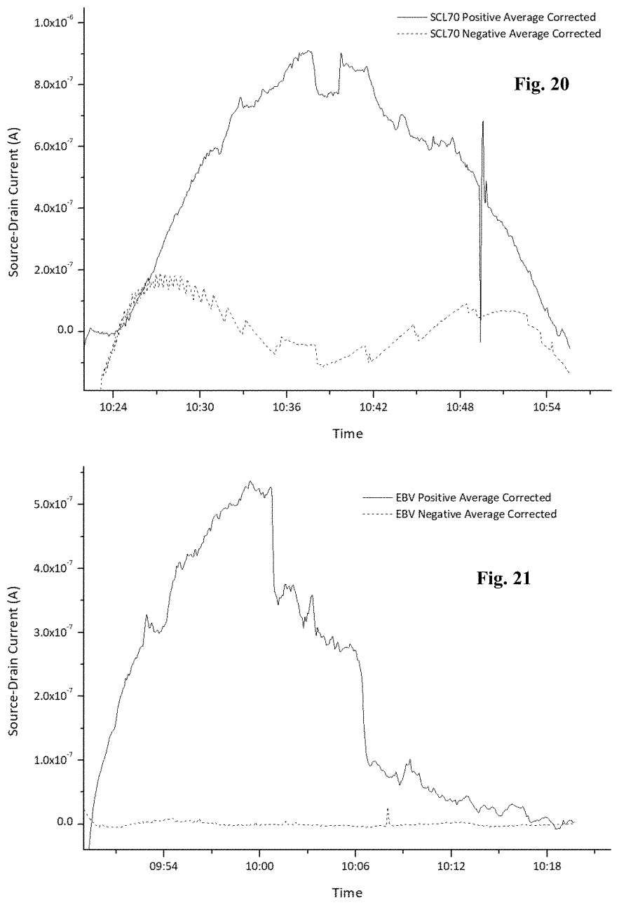

FIG. 19 shows the corrected plot averaged on four channels for the anti-testosterone hormone assay. FIG. 20 shows the corrected plot averaged on six channels for the SCL70 antibody assay. SCL 70 antibodies are considered to be specific for scleroderma (systemic sclerosis) and are found in up to 60% of patients with this connective tissue disease. In addition, SCL70 antibodies are considered to be a specific marker for the diffuse type of systemic sclerosis and also correlate with autoimmune disease. FIG. 21 shows the corrected plot averaged on ten channels for the EBV assay. This is the assay for the Epstein-Barr virus (EBV), which is one of eight known human herpesvirus types in the herpes family and is one of the most common viruses in humans. It is best known as the cause of infectious mononucleosis, such as mono or glandular fever.

### CONCLUSION

Charges formed in a liquid medium sensed by ISFET-type sensors come from the dissolved molecules. Depending on the pH value of the liquid and the molecules' isoelectric point, the dissolved molecules exhibit a global charge. However, this charge may be non-uniformly distributed over the molecule. In addition, the molecules have different sizes and a different 3D structure. Therefore, it is very important that:
(A) the sensor's interface is chemically engineered in a very uniform and reproducible manner,
(B) receptors need to be immobilised on the sensor's surface as highly selective receptor layer with a very uniform grafting density,
(C) the sensor should have redundant structure exhibiting multiple sensors cancelling out wrongly functionalised transistors, and
(D) a molecular friendly surface architecture and microenvironment with fixed pH value, fixed ionic strength and temperature needs to be established to avoid denaturation of the molecules on the sensor surface.
   The latter is controlled by respective reference sensors for temperature, pH and ionic strength in the sensor chip design. However, even with the above-mentioned ideal sensor design it can be the case that the potentiometric detection of charges, which lead to changes in surface potential and hence, to a shift of the ISFET threshold voltage, cannot be detected, because the relevant charges are located outside the Debye screening length of the liquid electrolyte.

The sensor of the present invention is different in all the aforementioned aspects. As noted above, it has recently and surprisingly been found by the present inventors that the PC-HEMT of the invention is capable of overcoming the Debye length limitation. The overall design of the PC-HEMT enables its additional operation in the frequency domain and helps to stabilise the electronic readout when recording even very small DC changes. Therefore, the sensor based on the PC-HEMT of the present invention can be used in impedance spectroscopy. Combination of potentiometric and impedimetric readout enables a more reliable sensing of molecules with the potential to sense beyond the Debye screening of electrical charges in an electrolyte solution, which is usually the limiting factor in most of the sensors having only potentiometric or conductometric readout.

As mentioned above, the PC-HEMT of the present invention can be optionally functionalised with different molecules (receptors), which are capable of binding to a target (analyte) molecule, for sensing. As a result, the PC-HEMT-based sensor of the invention can be used for label-free detection of target (analyte) molecules by monitoring changes in the electric current of the transistor caused by variations in the charge density or the impedance at the open gate-electrolyte interface. However, a more interesting approach is when the PC-HEMT is not functionalised, but still capable of sensing target molecules or biomolecules. As discussed above, sensing of the electric charges with the transistor of the present invention is possible in a contactless manner, when the molecules are at some distance from the surface of the transistor. This clearly allows to overcome a usual “sensing noise” of any traditional biosensor having reporter molecules attached to the surface of the sensor. Thus, the PC-HEMT-based sensor of the present invention can overcome the Debye-length limitation and tremendously increase the signal-to-noise ratio of the sensor and consequently, enhance sensitivity.

Moreover, the PC-HEMT-based sensor of the present invention does not require any surface modification with reporter molecules or any plate treatment or washing procedures or protocols. The sensing can be performed directly and immediately after immersing the sensor into any media being tested. This is in huge contrast to modern bioassays, such as ELISA or qPCR, which require hours and multistep laboratory procedures to perform. In addition, while most of the modern bioassays are single-use and require utilisation of the used materials and plates, the present sensor can be easily and immediately re-used after washing.

The above examples of the electronic ELISA of the present invention clearly indicate that many different analytes can be successfully detected with the sensor of the present invention. The sensor can distinguish between positive and negative patient serum with ultra-high sensitivity without having it modified. The sensor indicates biological activity at the sensor surface and can measure different concentrations of an analyte.

### Clarification Regarding the PC-HEMT Sensors

All the known HEMT devices are classified today in two general types: normally-on, and normally-off. Applicant has now discovered a third type (PC-HEMT), which is both normally-on and normally-of at the same time. It is a resonance semiconductor device operating based on a quantum superposition principle, hence its incredible sensitivity (switching between on and off takes no time).

The PC-HEMT-based sensors of the present invention are characterized in that the thickness of the top layer of the heterojunction structure in the open gate area is 5-9 nanometres (nm) and the surface of said top layer has a roughness of 0.2 nm or less, wherein the combination of said thickness and said roughness of the top layer is suitable for creating a quantum electronic effect of operating said 2DEG or 2DHG channel simultaneously in both normally-on and normally-off operation modes of the channel, thereby making said transistor suitable for conducting electric current through said channel in a quantum well between normally-on and normally-off operation modes of the transistor.

The amplification of the electrical current in the PC-HEMTs observed under any small external effect is tremendous. Every tiny electrical potential or piezoelectric impact, or a single molecule charge, even remotely (from the distance up to half a meter) immediately destroys this quantum equilibrium and either closes the channel or opens it. This is the quantum resonance effect that was observed unexpectedly when Applicant was working on improving the surface flatness of the top layer. When 0.2 nm roughness was reached by recessing the top layer down to 5 nm, removing atom layer by atom layer with short plasma pulses (etching) using Applicant's proprietary technique (described herein), an unusually drastic increase of the signal was suddenly registered. When approximately 4 nm was reached, this effect disappeared and the transistor returned its normal characteristics as a normally-off HEMT.

Following this surprising experimentally observed phenomenon, Applicant tested various ranges of thickness and roughness of the top layer and arrived to the conclusion that this incredibly high amplification of the HEMT sensitivity occurs only in the range of 5-9 nm thickness of the top layer. However, in order to obtain and observe this hypersensitivity of the HEMT, the top layer surface should be flat having roughness 0.2 nm and less. If such low roughness is not reached, this quantum effect cannot be created and observed.

1. A microelectronic device for sorbent, immunosorbent or cell sorbent assay, or flow cytometry, comprising:
(a) a plurality of microelectronic sensors, wherein said microelectronic sensors are integrated into said device in rows and in columns, thereby forming an array, and each of said microelectronic sensors is connected to its dedicated electrical contact in a contact array;
(b) the contact array integrated within said microelectronic device;
(c) a row multiplexer connected to said contact array for addressing each and every sensor arranged in rows, selecting one of several analogue or digital input signals and forwarding the selected input into a single line;
(d) a column multiplexer connected to said contact array for addressing each and every sensor arranged in columns, selecting one of several analogue or digital input signals and forwarding the selected input into a single line; and
(e) an integrated circuit for storing and processing said signals;
characterised in that each of said microelectronic sensors comprises at least one open-gate pseudo-conductive high-electron mobility transistor (PC-HEMT), said transistor comprising:
1) a multilayer hetero-junction structure made of gallium nitride (GaN) and aluminium gallium nitride (AlGaN) single-crystalline or polycrystalline semiconductor materials, and deposited on a substrate layer or placed on free-standing membranes, said structure comprising at least one buffer layer and at least one barrier layer, said layers being stacked alternately;
2) a conducting channel comprising a two-dimensional electron gas (2DEG) or a two-dimensional hole gas (2DHG), formed at the interface between said buffer layer and said barrier layer, and upon applying a bias to said transistor, becoming capable of providing electron or hole current, respectively, in said transistor between source and drain contacts; and
3) the source and drain contacts connected to said 2DEG or 2DHG conducting channel and to electrical metallisations for connecting said transistor to an electric circuit;
said transistor is characterised in that the thickness of a top layer of said heterojunction structure in an open gate area of the transistor is 5-9 nanometres (nm) and the surface of said top layer has a roughness of 0.2 nm or less, wherein the combination of said thickness and said roughness of the top layer is suitable for creating a quantum electronic effect of operating said 2DEG or 2DHG channel simultaneously in both normally-on and normally-off operation modes of the channel, thereby making said transistor suitable for conducting electric current through said channel in a quantum well between normally-on and normally-off operation modes of the transistor.

2. The microelectronic device of claim 1, wherein said transistor further comprising a Vivaldi antenna electrode or a metamaterial electrode, said Vivaldi antenna electrode or said metamaterial electrode being placed on the top layer between said source and drain contact in the open gate area of the transistor and capable of detecting electrical signals in the frequency range of 30 GHz to 300 THz.

3. The microelectronic device of claim 1, wherein said transistor is not coated with a molecular or biomolecular layer and is capable of remotely detecting target (analyte) gases, chemical compounds or biomolecules from the environment.

4. The microelectronic device of claim 1, wherein said transistor further comprises at least one molecular or biomolecular layer immobilised within the open gate area of the transistor and capable of binding or adsorbing target (analyte) gases, chemical compounds or biomolecules from the environment.

5. The microelectronic device of claim 1, wherein said multilayer hetero-junction structure comprises either:
A. one AlGaN barrier layer at the top of the structure recessed in the open gate area to the thickness of 5-9 nm with the surface roughness of 0.2 nm or less, and one GaN buffer layer at the bottom of the structure; said layers having Ga-face polarity, thus forming the two-dimensional electron gas (2DEG) conducting channel in said GaN layer, close to the interface with said AlGaN layer; or
B. one GaN layer at the top of the structure recessed in the open gate area to the thickness of 5-9 nm with the surface roughness of 0.2 nm or less, one GaN buffer layer at the bottom of the structure, and one AlGaN barrier layer in between; said layers having the Ga-face polarity, thus forming the two-dimensional hole gas (2DHG) conducting channel in the top GaN layer, close to the interface with said AlGaN barrier layer; or
C. one GaN layer at the top of the structure recessed in the open gate area to the thickness of 5-9 nm with the surface roughness of 0.2 nm or less, one GaN buffer layer at the bottom of the structure, and one AlGaN barrier layer in between; said layers having the N-face polarity, thus forming the two-dimensional electron gas (2DEG) conducting channel in the top GaN layer, close to the interface with said AlGaN barrier layer; or
D. one AlGaN barrier layer at the top of the structure recessed in the open gate area to the thickness of 5-9 nm with the surface roughness of 0.2 nm or less, and one GaN buffer layer at the bottom of the structure; said layers having N-face polarity, thus forming the two-dimensional hole gas (2DHG) conducting channel in said GaN layer, close to the interface with said AlGaN layer.

6. The microelectronic device of claim 1, wherein said source and drain contacts are ohmic.

7. The microelectronic device of claim 1, wherein the electrical metallizations of the transistor are capacitively-coupled to the 2DEG or 2DHG conducting channel for inducing displacement currents, thus resulting in said source and drain contacts being non-ohmic.

8. The microelectronic device of claim 1, wherein said transistor further comprises a dielectric layer deposited on top of said multilayer hetero-junction structure.

9. The microelectronic device of claim 1, wherein said substrate is gallium nitride (GaN) having thickness of 0.5-2 μm.

10. The microelectronic device of claim 1, wherein said free-standing membranes, on which the multilayer hetero-junction structure of the transistor is placed, are free-standing columns of substrate composed of sapphire, silicon, silicon carbide, gallium nitride or aluminium nitride.

11. The microelectronic device of claim 1, wherein the thickness of the top layer of the transistor in the open gate area is 6-7 nm; and the surface of said top layer has a roughness of 0.2 nm or less.

12. The microelectronic device of claim 11, wherein the thickness of said top layer in said open gate area is 6.2 nm to 6.4 nm.

13. The microelectronic device of claim 1, wherein said top layer has the roughness of about 0.1 nm or less, or 0.05 nm or less.

14. The microelectronic device of claim 1, wherein the multilayer heterojunction structure further comprises a piezoelectric electro-optical crystal (EOC) transducer adapted to be brought into a contact with a medium to be sensed and adapted to be illuminated with a polarised light.

15. The microelectronic device of claim 4, wherein said molecular or biomolecular layer is a cyclodextrin, 2,2,3,3-tetrafluoropropyloxy-substituted phthalocyanine or their derivatives, or said molecular or biomolecular layer comprises capturing biological molecules, such as primary, secondary antibodies or fragments thereof against certain proteins to be detected, or their corresponding antigens, enzymes or their substrates, short peptides, specific DNA sequences, which are complimentary to the sequences of DNA to be detected, aptamers, receptor proteins or molecularly imprinted polymers.

16. The microelectronic device of claim 1, wherein said device is a microelectronic microwell plate and each of said microelectronic sensors is integrated at the bottom of its corresponding microwell of said microwell plate.

17. A microelectronic microwell plate for sorbent, immunosorbent or cell sorbent assay, or flow cytometry, said microwell plate comprising:
(a) a plurality of the microelectronic sensors of claim 1, wherein each said sensor is integrated at the bottom of its corresponding well of said microwell plate and connected to its dedicated electrical contact in a contact array;
(b) the contact array integrated at the top of said microwell plate;
(c) a row multiplexer connected to said contact array for addressing each and every sensor arranged in rows, selecting one of several analogue or digital input signals and forwarding the selected input into a single line;
(d) a column multiplexer connected to said contact array for addressing each and every sensor arranged in columns, selecting one of several analogue or digital input signals and forwarding the selected input into a single line; and
(e) an integrated circuit for storing and processing said signals.

18. A method for sorbent, immunosorbent or cell sorbent assay of a sample containing a chemical compound or a biological compound to be tested in a gas phase or in a liquid phase, said method comprising:
(1) Subjecting the sample to the microelectronic device of claim 1;
(2) Recording electrical signals received from said microelectronic device in a form of a source-drain electric current of the microelectronic sensor over time (I_{DS} dynamics);
(3) Transmitting the recorded signals from said microelectronic device to an external memory for further processing; and
(4) Converting the transmitted signals to digital signals and processing the digital signals in the external memory, comparing said I_{DS} dynamics with negative control chemical or biomolecular I_{DS} waveforms stored in the external memory, and extracting biochemical or biomolecular information from said waveforms in a form of readable data, thereby detecting and/or identifying a particular biological compound or cell in the sample and measuring their concentration or amount and biochemical or biophysical parameters.

19. The method of claim 18, wherein said chemical compound or said biological compound is selected from the group of:
toxic metals, such as chromium, cadmium or lead,
regulated ozone-depleting chlorinated hydrocarbons,
food toxins, such as aflatoxin, and shellfish poisoning toxins, such as saxitoxin or microcystin,
neurotoxic compounds, such as methanol, manganese glutamate, nitrix oxide, tetanus toxin or tetrodotoxin, Botox, oxybenzone, Bisphenol A, or butylated hydroxyanisole,
explosives, such as picrates, nitrates, trinitro derivatives, such as 2,4,6-trinitrotoluene (TNT), 1,3,5-trinitro-1,3,5-triazinane (RDX), trinitroglycerine, N-methyl-N-(2,4,6-trinitrophenyl)nitramide (nitramine or tetryl), pentaerythritol tetranitrate (PETN), nitric ester, azide, derivates of chloric and perchloric acids, fulminate, acetylide, and nitrogen rich compounds, such as tetrazene, octahydro-1,3,5,7-tetranitro-1,3,5,7-tetrazocine (HMX), peroxide, such as triacetone trioxide, C4 plastic explosive and ozonidesor, or an associated compound of said explosives, such as a decomposition gases or taggants, and
biological pathogens, such as a respiratory viral or bacterial pathogen, an airborne pathogen, a plant pathogen, a pathogen from infected animals or a human viral pathogen.

20. A method for in-vitro measurement of cell dynamics, said method comprising:
(1) Subjecting a cell culture to a surface of a microelectronic sensor or growing said cell culture directly on the surface of said sensor;
(2) Recording electrical signals received from said microelectronic sensor in a form of a source-drain electric current of the microelectronic sensor over time (I_{D}s dynamics) in real time;
(3) Transmitting the recorded electrical signals from said microelectronic sensor to an external memory for further processing; and
(4) Converting the transmitted signals to digital signals and processing the digital signals in the external memory, comparing said I_{DS} dynamics with negative control I_{DS} waveforms stored in the external memory, and extracting information on cell dynamics and cell contractility from said waveforms in a form of readable data;
characterised in that said microelectronic sensor comprises at least one open-gate pseudo-conductive high-electron mobility transistor (PC-HEMT), said transistor comprising:
1) a multilayer hetero-junction structure made of gallium nitride (GaN) and aluminium gallium nitride (AlGaN) single-crystalline or polycrystalline semiconductor materials, and deposited on a substrate layer or placed on free-standing membranes, said structure comprising at least one buffer layer and at least one barrier layer, said layers being stacked alternately;
2) a conducting channel comprising a two-dimensional electron gas (2DEG) or a two-dimensional hole gas (2DHG), formed at the interface between said buffer layer and said barrier layer, and upon applying a bias to said transistor, becoming capable of providing electron or hole current, respectively, in said transistor between source and drain contacts; and
3) the source and drain contacts connected to said 2DEG or 2DHG conducting channel and to electrical metallisations for connecting said transistor to an electric circuit;
said transistor is characterised in that the thickness of a top layer of said heterojunction structure in an open gate area of the transistor is 5-9 nanometres (nm) and the surface of said top layer has a roughness of 0.2 nm or less, wherein the combination of said thickness and said roughness of the top layer is suitable for creating a quantum electronic effect of operating said 2DEG or 2DHG channel simultaneously in both normally-on and normally-off operation modes of the channel, thereby making said transistor suitable for conducting electric current through said channel in a quantum well between normally-on and normally-off operation modes of the transistor.
